(19) United States
(12) Patent Application Publication
(10) Pub. No.: US 2022/0223259 A1
(43) Pub. Date: Jul. 14, 2022

(54) Risk Assessment for Suicide and Treatment Based on Interaction with Virtual Clinician, Food Intake Tracking, and/or Satiety Determination

(71) Applicants: Cecilia BERGH, Stockholm (SE); Per SÖDERSTEN, Stockholm (SE); Jenny VAN DEN BOSSCHE NOLSTAM, Stockholm (SE); Ulf BRODIN, Stockholm (SE); Modjtaba ZANDIAN, Stockholm (SE); Michael LEON, San Juan Capistrano, CA (US); Mandometer AB, Huddinge (SE)

(72) Inventors: Cecilia Bergh, Stockholm (SE); Per Södersten, Stockholm (SE); Jenny Van den Bossche Nolstam, Stockholm (SE); Ulf Brodin, Stockholm (SE); Modjtaba Zandian, Stockholm (SE); Michael Leon, San Juan Capistrano, CA (US)

(21) Appl. No.: 17/611,799

(22) PCT Filed: Apr. 29, 2020
(86) PCT No.: PCT/US20/30372

§ 371 Date: Nov. 16, 2021

Related U.S. Application Data
(60) Provisional application No. 62/851,238, filed on May 22, 2019.

(51) Int. Cl. G16H 20/70; G16H 20/60; G16H 10/60; G16H 80/00; G16H 10/20; G06F 40/35

(52) U.S. Cl. CPC G16H 20/70; G16H 20/60; G06F 40/35; G16H 80/00; G16H 10/20; G16H 10/60

(57) ABSTRACT

Novel tools and techniques are provided for implementing medical or medical-related diagnosis and treatment, and, more particularly, for implementing risk assessment for suicide and treatment based on interaction with virtual clinician, food intake tracking, and/or satiety determination. In various embodiments, a computing system might generate a virtual clinician capable of simulating facial expressions and body expressions, and might cause, using a display device and/or an audio output device, the generated virtual clinician to interact with a patient. The computing system might analyze the interactions between the virtual clinician and the patient (and in some cases, food intake data) to determine likelihood of risk of suicide by the patient, and, based on a determination that a likelihood of risk of suicide by the patient exceeds a first predetermined threshold value, might send an alert message to one or more healthcare professionals regarding the likelihood of risk of suicide by the patient.

### CROSS-REFERENCES TO RELATED APPLICATIONS

This application claims priority to U.S. Patent Application Ser. No. 62/851,238 (the “'238 Application”), filed May 22, 2019 by Cecilia Bergh et al. (attorney docket no. 1115.03PR), entitled, “Method and System for Implementing Risk Assessment for Suicide and Treatment Based on Interaction with Virtual Clinician, Food Intake Tracking, and/or Satiety Determination,” the disclosure of which is incorporated herein by reference in its entirety for all purposes.

This application may be related to U.S. patent application Ser. No. 12/412,434 (the “'434 application”; U.S. Pat. No. 10,332,054) filed Mar. 27, 2009 by Cecilia Bergh (attorney docket no. 1115.02), entitled, “Method, Generator Device, Computer Program Product and System for Generating Medical Advice,” which claims priority to SE Application No. 0900156-1, filed Feb. 9, 2009, by Cecilia Bergh, the disclosure of which is incorporated herein by reference in its entirety for all purposes.

The respective disclosures of these applications/patents (which this document refers to collectively as the “Related Applications”) are incorporated herein by reference in their entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to methods, systems, and apparatuses for implementing medical or medical-related diagnosis and treatment, and, more particularly, to methods, systems, and apparatuses for implementing risk assessment for suicide and treatment based on interaction with virtual clinician, food intake tracking, and/or satiety determination.

### BACKGROUND

Conventional human physician techniques are unable to accurately, precisely, and consistently identify or diagnosis suicidal thoughts and tendencies in patients. The inventor is also unaware of conventional virtual clinicians that are capable of performing the tasks of human physicians in this regard, much less surpassing the capabilities of human physicians. Moreover, conventional techniques, whether performed by human physicians or by conventional virtual clinicians, fail to take into account food intake and satiety as factors to consider when analyzing to determine likelihood of risk of suicide by the patient.

Hence, there is a need for more robust and scalable solutions for implementing medical or medical-related diagnosis and treatment, and, more particularly, to methods, systems, and apparatuses for implementing risk assessment for suicide and treatment based on interaction with virtual clinician, food intake tracking, and/or satiety determination.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic diagram illustrating a system for implementing risk assessment for suicide and treatment based on interaction with virtual clinician, food intake tracking, and/or satiety determination, in accordance with various embodiments.

FIG. 2A is a schematic diagram illustrating a non-limiting example of flagged words and expressions that are stored in a datastore and that may be used to implement risk assessment for suicide and treatment, in accordance with various embodiments

FIGS. 2B-2E are schematic diagrams illustrating a non-limiting example of various interfaces that may be used for implementing risk assessment for suicide and treatment based on interaction with virtual clinician, food intake tracking, and/or satiety determination may be implemented, in accordance with various embodiments.

FIGS. 3A and 3B are schematic block flow diagrams illustrating a method for implementing risk assessment for suicide and treatment based on interaction with virtual clinician, food intake tracking, and/or satiety determination, in accordance with various embodiments.

FIGS. 4A-4E are flow diagrams illustrating a method for implementing risk assessment for suicide and treatment based on interaction with virtual clinician, food intake tracking, and/or satiety determination, in accordance with various embodiments.

FIG. 5 is a block diagram illustrating an exemplary computer or system hardware architecture, in accordance with various embodiments.

FIG. 6 is a block diagram illustrating a networked system of computers, computing systems, or system hardware architecture, which can be used in accordance with various embodiments.

### DETAILED DESCRIPTION OF CERTAIN EMBODIMENTS

Overview

Various embodiments provide tools and techniques for implementing medical or medical-related diagnosis and treatment, and, more particularly, to methods, systems, and apparatuses for implementing risk assessment for suicide and treatment based on interaction with virtual clinician, food intake tracking, and/or satiety determination.

In various embodiments, a computing system might generate a virtual clinician capable of simulating facial expressions and body expressions, and might cause, using a display device and/or an audio output device, the generated virtual clinician to interact with a patient, via at least one of participating in a conversation with the patient, asking the patient one or more questions, or answering one or more questions posed by the patient, wherein interactions between the virtual clinician and the patient might be based at least in part on one or more of words, verbal expressions, statements, sentences, sentence responses, questions, or answers that are stored in a database, and/or the like. The computing system might record, to a datastore, interactions between the virtual clinician and the patient.

The computing system, using the display device viewable by the patient and/or the audio output device, might prompt the patient to select a facial expression among a range of facial expressions that represents current emotions of the patient, and might receive a first response from the patient, the first response comprising a selection of a facial expression that represents current emotions of the patient. Alternatively, or additionally, the computing system, using the display device viewable by the patient and/or the audio output device, might prompt the patient to select a body posture among a range of body postures that represents current emotions of the patient, and might receive a second response from the patient, the second response comprising a selection of a body posture that represents current emotions of the patient. Alternatively, or additionally, the computing system, using the display device viewable by the patient and/or the audio output device, might prompt the patient to select a statement regarding zest for life among a range of statements regarding zest for life that represents current thoughts of the patient regarding life and death, and might receive a third response from the patient, the third response comprising a selection of a statement regarding zest for life that represents current thoughts of the patient regarding life and death.

Alternatively, or additionally, the computing system might receive food intake and satiety data associated with the patient, the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals, and/or the like.

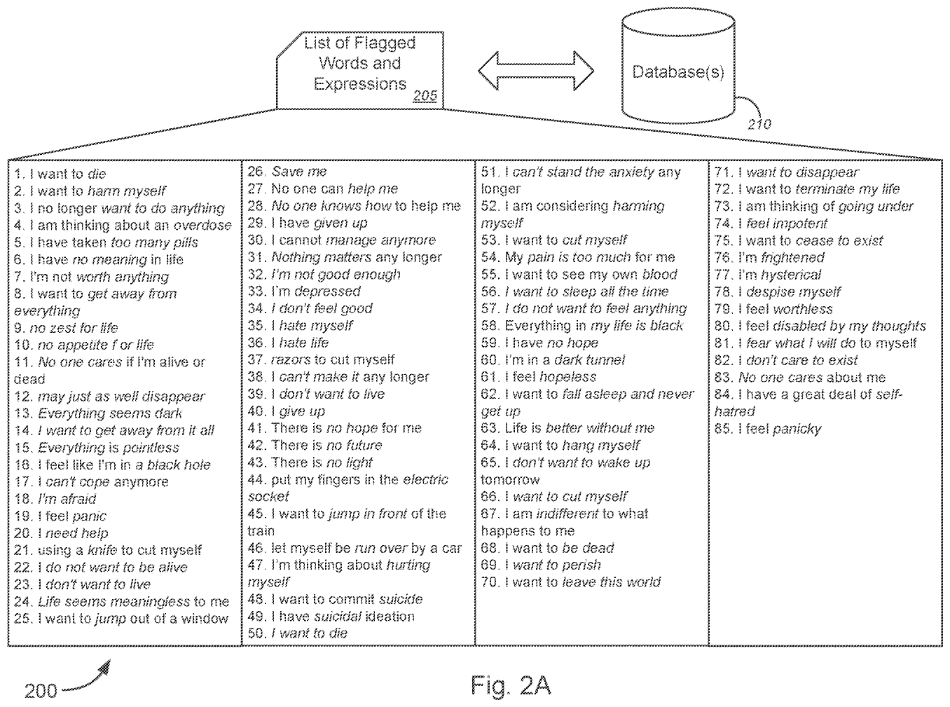

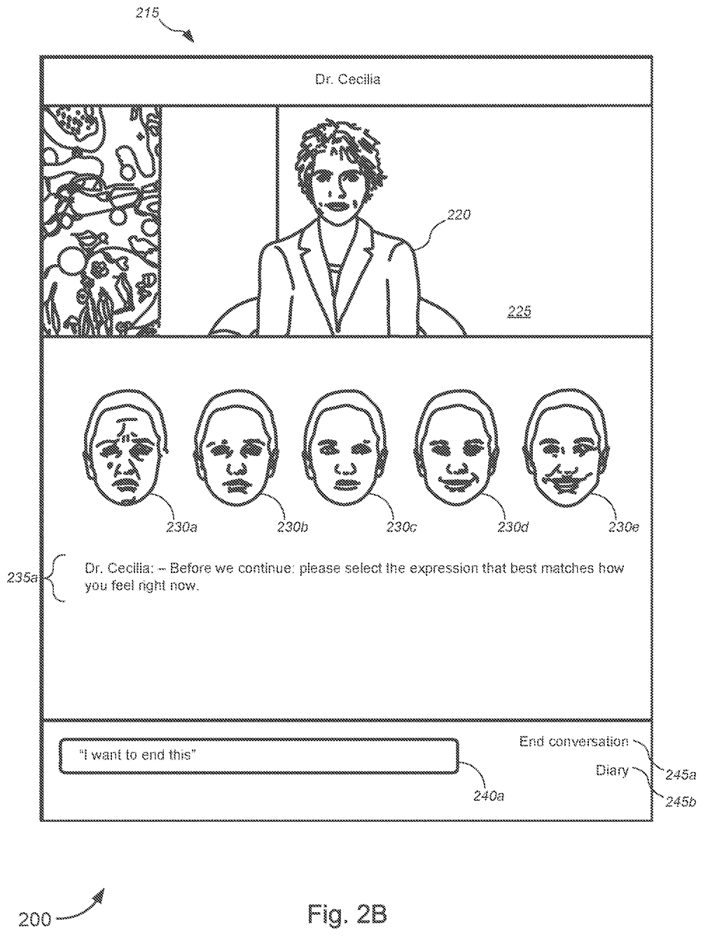

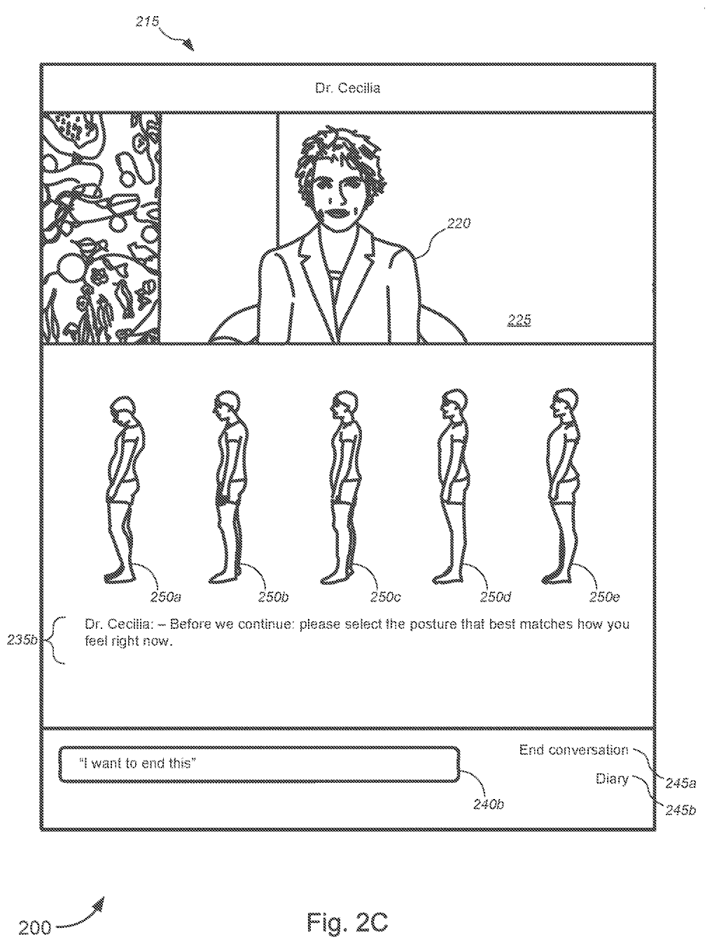

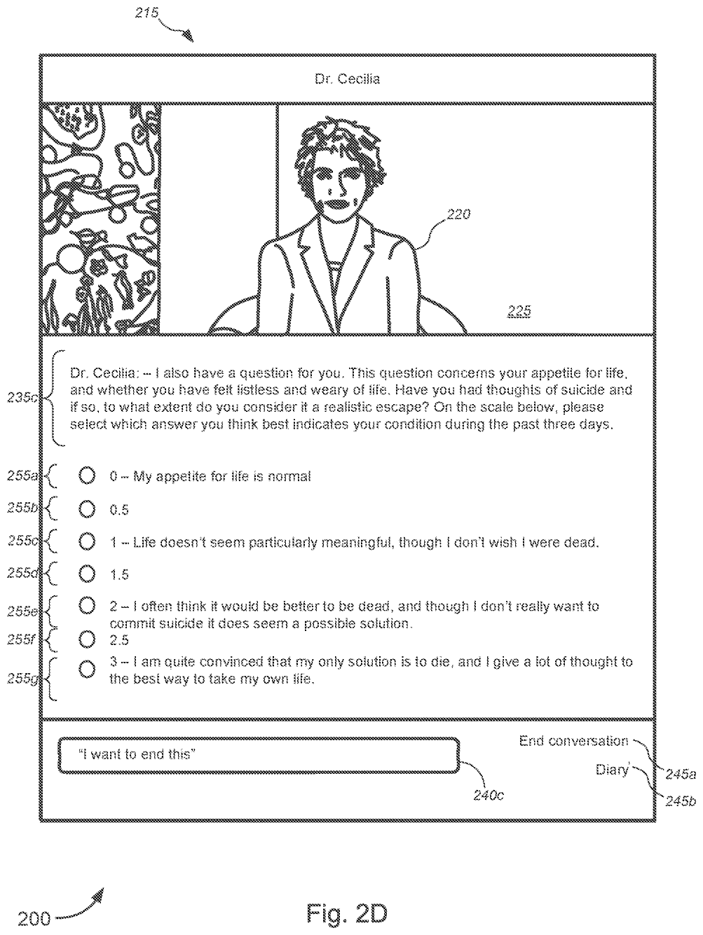

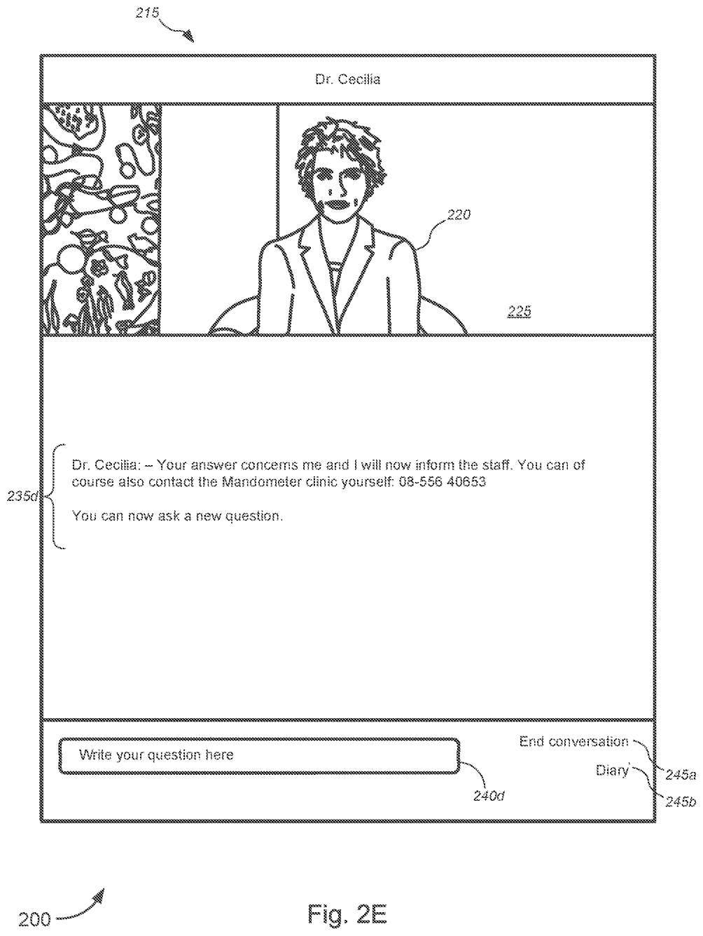

The computing system might analyze at least one of the recorded interactions between the virtual clinician and the patient, the received first response, the received second response, the received third response, or the received food intake and satiety data associated with the patient, to determine likelihood of risk of suicide by the patient. Part of the analysis of the recorded interactions between the virtual clinician and the patient might be to identify flagged words or expressions (as described herein, and as shown in FIG. 2A, for example). Based on a determination that a likelihood of risk of suicide by the patient exceeds a first predetermined threshold value, the computing system might send a message to one or more healthcare professionals regarding the likelihood of risk of suicide by the patient. Based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.

In some aspects, when interacting with a patient, the virtual clinician (i.e., Dr. Cecilia) might identify words and expressions indicating that the patient does not feel well and is about to harm himself, herself, or themselves (herein, referred to as “flagged words or expressions” or the like). A technique that may be used in identifying flagged words or expressions might include, without limitation, n-gram technique, which utilizes a contiguous sequence of a given number of items or words to identify flagged words or expressions. Examples of (a) a 1-gram (or unigram), (b) a 2-gram (or bigram), (c) a 3-gram (or trigram), or (d) a 4-gram of the expression, “I want to harm myself,” might be as follows: (a) “I,” “want,” “to,” “harm,” and “myself”; (b) “I want,” “want to,” “to harm,” and “harm myself”; (c) “I want to,” want to harm,” and “to harm myself”; and (4) “I want to harm” and “want to harm myself”; and so on. Analysis by using n-grams may facilitate identification of flagged words or expressions.

It has been found by the inventor that patients are more honest when interacting with a virtual clinician, such as Dr. Cecilia, than when interacting with a human clinician, and that computer software is better at the diagnosis of psychological problems than a human physician. When a patient is using a words, expression, or statement indicating that he, she, or they are at risk of suicide, the virtual clinician might ask the patient to select a facial expression that matches his, her, or their emotions, to select a posture that is consistent with that emotion, and to select from a list of statements that reflects the patient's zest for life. From the numerical scale shown in FIG. 2D, for example, if the patient scores 2 or higher on the 0-3 emotion scale, an e-mail, SMS, or a telephone signal might alert the physician on duty. The conversation or interaction that the patient had with Dr. Cecilia might then be forwarded to the physician, including the frequency of words indicative of risk of suicide over the previous three days.

As discussed herein, the system identifies five measurable concepts that may be tracked, including, for example: (A) flagged words, or expressions (such as shown in the non-limiting example of FIG. 2A); (B) facial expressions (such as shown in the non-limiting example of FIG. 2B); (C) postures (such as shown in the non-limiting example of FIG. 2C); (D) 15 subjects or topics consisting of categorized answers from Dr. Cecilia to questions asked by patients; and (E) eating behavior measured by scale or the like. Regarding (D), the subjects or topics might be classified from “healthy” to “severely ill,” and the answers given by Dr. Cecilia are made to coincide with generation of facial expression on the face of the virtual clinician, Dr. Cecilia, to reflect corresponding concerned, serious, neutral, positive, and/or happy emotions. Regarding (D), the 15 subjects or topics include, but are not limited to, (1) the illness, (2) fear of fatness, (3) striving for thinness, (4) bulimia, (5a) health consequences—psychiatric (e.g., anxiety, compulsion, depression, mood changes, dark thoughts, etc.), (5b) health consequences—physical, (6) social consequences, (7) the treatment (e.g., anxiety reduction, thermal treatment, reward schedule, physical activity, eating behavior and satiety, Mandometer training or training with scale and other tools, food schedule, forbidden foods, etc.), (8) eating behavior and satiety, (9) physical activity, (10) weight, (11) food, (12) social reconstruction, (13) healthcare professionals, (14) remission, and (15) relapse, or the like. Regarding (E), the concept of eating behavior might include three variables: (I) the deviation from a normal meal; (II) eating pattern (whether linear or decelerated eating); and (III) occurrence of displaced behaviors during a meal (e.g., reheating the meal to prolong duration of the meal, which is a typical anorectic behavior).

In another aspect, the system might be configured to treat the patient, by helping patient eat food in a manner that would allow release of satiety hormones in the patient's body to evoke a normal feeling of fullness, by providing audible and/or visual cues prompting the patient to eat either faster or slower if the system determines that the patient is eating too slow or too fast. The system registers eating rate (measured in grams per minute, or the like), amount of food (measured in grams, or the like), and duration of each meal (measured in minutes). A normal meal might consist of 300-350 grams eaten over 12-15 minutes. A healthy subject would display a decelerated eating behavior—that is, eating fast at the beginning of the meal, then slowing near the end of the meal. On the other hand, a subject with an eating disorder or who is considered obese might display a linear eating behavior—that is, eating at the same pace throughout the meal. Subjects with eating disorders or who are considered obese have been found by the inventor to be more likely than healthy subjects to exhibit sad or very sad feelings and to harbor thoughts of suicide. As such, these food intake data are useful as a factor to analyze to determine likelihood of suicide. The system might also prompt the patient at regular intervals to record his, her, or their feelings of fullness (or satiety) (e.g., slight, moderate, strong, very strong, or extreme, or the like). Such satiety data is also useful as another factor to analyze to determine likelihood of suicide.

In terms of treatment of patients, each interaction or session that the patient has with the virtual clinician might be described as a process measuring intensity of concepts and time. The processes of patients in treatment might be compared to the same variables for patients with a successful outcome (i.e., patients in remission or recovery, or the like). The processes might affect each other in time series. By dichotomizing the individual responses into sets of patients and non-patients, one can generate a graphs of the risk behavior for healthy vs. unhealthy individuals. For example, a successful treatment of a patient might show the number or intensity of flagged words or expressions starting high, then decreasing over the course of days, weeks, or months. Whereas, an unsuccessful treatment of a patient might show the number or intensity of flagged words or expressions remaining substantially unchanged over the course of days, weeks, or months. In some embodiments, treatment of the patient might include, without limitation, interactions with the patient that promote more decelerated eating behaviors, and/or interactions with the patient that aim to discover the source(s) of sadness or depression in the patient and suggesting ways to address or overcome these underlying issues, and/or interactions with the patient that aim to discover positive aspects of the patient's life and suggesting ways for the patient to focus on those positive aspects, and/or the like.

These and other aspects of the risk assessment for suicide and treatment based on interaction with virtual clinician, food intake tracking, and/or satiety determination are described in greater detail with respect to the figures.

The following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Various embodiments described herein, while embodying (in some cases) software products, computer-performed methods, and/or computer systems, represent tangible, concrete improvements to existing technological areas, including, without limitation, medical diagnosis technology, medical-related diagnosis technology, medical diagnosis and treatment technology, medical-related diagnosis and treatment technology, virtual human interface technology, and/or the like. In other aspects, certain embodiments, can improve the functioning of user equipment or systems themselves (e.g., medical diagnosis systems, medical-related diagnosis systems, medical diagnosis and treatment systems, medical-related diagnosis and treatment systems, virtual human interface systems, etc.), for example, by generating, with a computing system, a virtual clinician capable of simulating facial expressions and body expressions; causing, with the computing system, the generated virtual clinician to interact with a patient; analyzing, with the computing system, the interactions between the virtual clinician and the patient to determine likelihood of risk of suicide by the patient; and based on a determination that a likelihood of risk of suicide by the patient exceeds a first predetermined threshold value, sending, with the computing system, an alert message to one or more healthcare professionals regarding the likelihood of risk of suicide by the patient; and/or the like.

In particular, to the extent any abstract concepts are present in the various embodiments, those concepts can be implemented as described herein by devices, software, systems, and methods that involve specific novel functionality (e.g., steps or operations), such as, generating, with a computing system, a virtual clinician capable of simulating facial expressions and body expressions; causing, with the computing system, the generated virtual clinician to interact with a patient; analyzing, with the computing system, the interactions between the virtual clinician and the patient to determine likelihood of risk of suicide by the patient; and based on a determination that a likelihood of risk of suicide by the patient exceeds a first predetermined threshold value, sending, with the computing system, an alert message to one or more healthcare professionals regarding the likelihood of risk of suicide by the patient; and/or the like, to name a few examples, that extend beyond mere conventional computer processing operations. These functionalities can produce tangible results outside of the implementing computer system, including, merely by way of example, optimized interaction with users using a generated virtual clinician and diagnosis and treatment of suicidal thoughts and tendencies based on such optimized interaction, and/or the like, at least some of which may be observed or measured by customers and/or service providers.

In an aspect, a method might comprise generating, with a computing system, a virtual clinician capable of simulating facial expressions and body expressions; causing, with the computing system and using a display device and an audio output device, the generated virtual clinician to interact with a patient, via at least one of participating in a conversation with the patient, asking the patient one or more questions, or answering one or more questions posed by the patient, wherein interactions between the virtual clinician and the patient are based at least in part on one or more of words, verbal expressions, statements, sentences, sentence responses, questions, or answers that are stored in a database; and recording, with the computing system and to a datastore, interactions between the virtual clinician and the patient. The method might further comprise prompting the patient, with the computing system and using the display device viewable by the patient and the audio output device, to select a facial expression among a range of facial expressions that represents current emotions of the patient; receiving, with the computing system, a first response from the patient, the first response comprising a selection of a facial expression that represents current emotions of the patient; prompting the patient, with the computing system and using the display device viewable by the patient and the audio output device, to select a body posture among a range of body postures that represents current emotions of the patient; receiving, with the computing system, a second response from the patient, the second response comprising a selection of a body posture that represents current emotions of the patient; prompting the patient, with the computing system and using the display device viewable by the patient and the audio output device, to select a statement regarding zest for life among a range of statements regarding zest for life that represents current thoughts of the patient regarding life and death; and receiving, with the computing system, a third response from the patient, the third response comprising a selection of a statement regarding zest for life that represents current thoughts of the patient regarding life and death.

The method might also comprise receiving, with the computing system, food intake and satiety data associated with the patient, the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals. The method might further comprise analyzing, with the computing system, at least one of the recorded interactions between the virtual clinician and the patient, the received first response, the received second response, the received third response, or the received food intake and satiety data associated with the patient, to determine likelihood of risk of suicide by the patient; based on a determination that a likelihood of risk of suicide by the patient exceeds a first predetermined threshold value, sending, with the computing system, a message to one or more healthcare professionals regarding the likelihood of risk of suicide by the patient; and based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.

In some embodiments, the computing system might comprise at least one of a tablet computer, a laptop computer, a desktop computer, a local server, a dedicated food intake tracking device, a user device, a server computer over a network, or a cloud-based computing system over a network, and/or the like.

In another aspect, a method might comprise generating, with a computing system, a virtual clinician capable of simulating facial expressions and body expressions; causing, with the computing system, the generated virtual clinician to interact with a patient; analyzing, with the computing system, the interactions between the virtual clinician and the patient to determine likelihood of risk of suicide by the patient; and based on a determination that a likelihood of risk of suicide by the patient exceeds a first predetermined threshold value, sending, with the computing system, an alert message to one or more healthcare professionals regarding the likelihood of risk of suicide by the patient.

In some embodiments, causing the generated virtual clinician to interact with the patient might comprise causing, with the computing system, the generated virtual clinician to interact with a patient, via at least one of participating in a conversation with the patient, asking the patient one or more questions, or answering one or more questions posed by the patient, and/or the like. In some cases, interactions between the virtual clinician and the patient might be based at least in part on one or more of using, recognizing, or interpreting one or more of words, verbal expressions, statements, sentences, sentence responses, questions, or answers, and/or the like, that are stored in a database.

According to some embodiments, the method might comprise recording, with the computing system and to a datastore, interactions between the virtual clinician and the patient. In some instances, recording the interactions between the virtual clinician and the patient and analyzing the interactions between the virtual clinician and the patient to determine likelihood of risk of suicide by the patient might be performed in real-time or near-real-time.

In some embodiments, causing the generated virtual clinician to interact with the patient might comprise one of: interacting with the patient by displaying the generated virtual clinician on a display device and displaying words of the virtual clinician as text on the display device; interacting with the patient by displaying the generated virtual clinician on a display device and presenting words of the virtual clinician via an audio output device; or interacting with the patient by displaying the generated virtual clinician on a display device, presenting words of the virtual clinician via an audio output device, and displaying words of the virtual clinician as text on the display device; or the like.

Alternatively, or additionally, causing the generated virtual clinician to interact with a patient might comprise at least one of: prompting the patient, with the computing system, to select a facial expression among a range of facial expressions that represents current emotions of the patient, and receiving, with the computing system, a first response from the patient, the first response comprising a selection of a facial expression that represents current emotions of the patient; prompting the patient, with the computing system, to select a body posture among a range of body postures that represents current emotions of the patient, and receiving, with the computing system, a second response from the patient, the first response comprising a selection of a body posture that represents current emotions of the patient; or prompting the patient, with the computing system, to select a statement regarding zest for life among a range of statements regarding zest for life that represents current thoughts of the patient regarding life and death, and receiving, with the computing system, a third response from the patient, the third response comprising a selection of a statement regarding zest for life that represents current thoughts of the patient regarding life and death; and/or the like. In such cases, analyzing the interactions between the virtual clinician and the patient to determine likelihood of risk of suicide by the patient might comprise analyzing, with the computing system, the interactions between the virtual clinician and the patient and at least one of the received first response, the received second response, or the received third response, and/or the like, to determine likelihood of risk of suicide by the patient.

Alternatively, or additionally, causing the generated virtual clinician to interact with the patient might comprise at least one of: recording video of the patient during the interaction and utilizing at least one of facial analysis, body analysis, or speech analysis to identify at least one of facial expressions of the patient, body language of the patient, or words spoken by the patient; recording audio of the patient during the interaction and utilizing speech analysis to identify words spoken by the patient; or recording words typed by the patient via a user interface device and utilizing text analysis to identify words typed by the patient; and/or the like.

According to some embodiments, analyzing the interactions between the virtual clinician and the patient to determine likelihood of risk of suicide by the patient might comprise determining, with the computing system, whether words or expressions spoken or typed by the patient match predetermined flagged words and expressions that are indicative of suicide risk, and determining, with the computing system, likelihood of risk of suicide by the patient, based at least in part on a determination that words or expressions spoken or typed by the patient match predetermined flagged words and expressions that are indicative of suicide risk.

Alternatively, or additionally, analyzing the interactions between the virtual clinician and the patient to determine likelihood of risk of suicide by the patient might comprise analyzing, with the computing system, the interactions between the virtual clinician and the patient and historical data associated with the patient to determine likelihood of risk of suicide by the patient. In some cases, the historical data might comprise at least one of interactions between the virtual clinician and the patient during one or more prior sessions, one or more diary entries entered by the patient, one or more records containing words or expressions previous spoken or typed by the patient that match predetermined flagged words and expression that are indicative of suicide risk, one or more records containing data related to emotions of the patient during prior sessions, one or more prior suicide risk assessments for the patient, or one or more prior medical-related assessments performed on the patient, and/or the like.

Alternatively, or additionally, analyzing the interactions between the virtual clinician and the patient to determine likelihood of risk of suicide by the patient might comprise utilizing at least one of artificial intelligence functionality or machine learning functionality to determine likelihood of risk of suicide by the patient.

In some embodiments, the method might further comprise receiving, with the computing system, food intake and satiety data associated with the patient. In some cases, the food intake and satiety data might comprise at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals, and/or the like. In such cases, analyzing the interactions between the virtual clinician and the patient to determine likelihood of risk of suicide by the patient might comprise analyzing, with the computing system, at least one of the interactions between the virtual clinician and the patient or the received food intake and satiety data associated with the patient, to determine likelihood of risk of suicide by the patient.

In some instances, the food intake and satiety data associated with the patient might be received from at least one of a communications-enabled scale that is used to measure weight of food on a food container during meals consumed by the patient where the food is consumed out of the food container during the meals, a user device that is communicatively coupled to the communications-enabled scale, or the user device that records self-reported feelings of satiety from the patient during meals, and/or the like. In some cases, the method might further comprise, based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations, and/or the like, that correspond to levels designed to stimulate physiological responses that evoke positive feelings for the patient.

In yet another aspect, an apparatus might comprise at least one processor and a non-transitory computer readable medium communicatively coupled to the at least one processor. The non-transitory computer readable medium might have stored thereon computer software comprising a set of instructions that, when executed by the at least one processor, causes the apparatus to: generate a virtual clinician capable of simulating facial expressions and body expressions; cause the generated virtual clinician to interact with a patient; analyze the interactions between the virtual clinician and the patient to determine likelihood of risk of suicide by the patient; and based on a determination that a likelihood of risk of suicide by the patient exceeds a first predetermined threshold value, send an alert message to one or more healthcare professionals regarding the likelihood of risk of suicide by the patient.

In some embodiments, causing the generated virtual clinician to interact with the patient might comprise causing the generated virtual clinician to interact with a patient, via at least one of participating in a conversation with the patient, asking the patient one or more questions, or answering one or more questions posed by the patient, and/or the like. In some cases, interactions between the virtual clinician and the patient might be based at least in part on one or more of words, verbal expressions, statements, sentences, sentence responses, questions, or answers, and/or the like, that are stored in a database.

According to some embodiments, analyzing the interactions between the virtual clinician and the patient to determine likelihood of risk of suicide by the patient might comprise determining whether words or expressions spoken or typed by the patient match predetermined flagged words and expressions that are indicative of suicide risk, and determining likelihood of risk of suicide by the patient, based at least in part on a determination that words or expressions spoken or typed by the patient match predetermined flagged words and expressions that are indicative of suicide risk.

In some embodiments, the set of instructions, when executed by the at least one processor, might further cause the apparatus to: receive food intake and satiety data associated with the patient, the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals. In such cases, analyzing the interactions between the virtual clinician and the patient to determine likelihood of risk of suicide by the patient might comprise analyzing at least one of the interactions between the virtual clinician and the patient or the received food intake and satiety data associated with the patient, to determine likelihood of risk of suicide by the patient.

In still another aspect, a system might comprise a computing system, which might comprise at least one first processor and a first non-transitory computer readable medium communicatively coupled to the at least one first processor. The first non-transitory computer readable medium might have stored thereon computer software comprising a first set of instructions that, when executed by the at least one first processor, causes the computing system to: generate a virtual clinician capable of simulating facial expressions and body expressions; cause the generated virtual clinician to interact with a patient; analyze the interactions between the virtual clinician and the patient to determine likelihood of risk of suicide by the patient; and based on a determination that a likelihood of risk of suicide by the patient exceeds a first predetermined threshold value, send an alert message to one or more healthcare professionals regarding the likelihood of risk of suicide by the patient.

According to some embodiments, the system might further comprise a scale that is used to measure weight of food on a food container during meals consumed by the patient, where the food is consumed out of the food container during the meals, and a user device associated with the user, and communicatively coupled to the scale. The user device might comprise at least one second processor and a second non-transitory computer readable medium communicatively coupled to the at least one second processor. The second non-transitory computer readable medium might have stored thereon computer software comprising a second set of instructions that, when executed by the at least one second processor, causes the user device to: receive food intake data associated with the patient from the scale, wherein the food intake and satiety data comprises at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, or information regarding occurrence of any displaced behaviors during a meal; prompt the patient to enter self-reported feelings of satiety from the patient during meals and receive satiety data from the patient; and send food intake and satiety data associated with the patient to the computing system. In some cases, analyzing the interactions between the virtual clinician and the patient to determine likelihood of risk of suicide by the patient might comprise analyzing at least one of the interactions between the virtual clinician and the patient or the received food intake and satiety data associated with the patient, to determine likelihood of risk of suicide by the patient.

In some instances, the user device comprises one of a tablet computer, a smart phone, a mobile phone, a laptop computer, a desktop computer, or a dedicated food intake tracking device, and/or the like.

In an aspect, a method might comprise receiving, with a computing system, food intake and satiety data associated with the patient; analyzing, with the computing system, the food intake and satiety data associated with the patient to determine likelihood of risk of suicide by the patient; and based on a determination that a likelihood of risk of suicide by the patient exceeds a first predetermined threshold value, sending, with the computing system, an alert message to one or more healthcare professionals regarding the likelihood of risk of suicide by the patient.

According to some embodiments, the computing system might comprise at least one of a tablet computer, a laptop computer, a desktop computer, a local server, a dedicated food intake tracking device, a user device, a server computer over a network, or a cloud-based computing system over a network.

In some embodiments, the food intake and satiety data might comprise at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals, and/or the like. In some cases, the food intake and satiety data associated with the patient might be received from at least one of a communications-enabled scale that is used to measure weight of food on a food container during meals consumed by the patient where the food is consumed out of the food container during the meals, a user device that is communicatively coupled to the communications-enabled scale, or the user device that records self-reported feelings of satiety from the patient during meals, and/or the like. In some instances, the user device might comprise one of a tablet computer, a smart phone, a mobile phone, a laptop computer, a desktop computer, or a dedicated food intake tracking device, and/or the like.

According to some embodiments, the method might further comprise based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.

In another aspect, a system might comprise a computing system, which might comprise at least one first processor and a first non-transitory computer readable medium communicatively coupled to the at least one first processor. The first non-transitory computer readable medium might have stored thereon computer software comprising a first set of instructions that, when executed by the at least one first processor, causes the computing system to: receive food intake and satiety data associated with a patient; analyze the food intake and satiety data associated with the patient to determine likelihood of risk of suicide by the patient; and based on a determination that a likelihood of risk of suicide by the patient exceeds a first predetermined threshold value, send an alert message to one or more healthcare professionals regarding the likelihood of risk of suicide by the patient.

In some embodiments, the computing system might comprise at least one of a tablet computer, a laptop computer, a desktop computer, a local server, a dedicated food intake tracking device, a user device, a server computer over a network, or a cloud-based computing system over a network, and/or the like. In some cases, the food intake and satiety data might comprise at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, or information regarding occurrence of any displaced behaviors during a meal, and/or the like. In some instances, the food intake and satiety data associated with the patient might be received from at least one of a communications-enabled scale that is used to measure weight of food on a food container during meals consumed by the patient where the food is consumed out of the food container during the meals, a user device that is communicatively coupled to the communications-enabled scale, or the user device that records self-reported feelings of satiety from the patient during meals, and/or the like.

According to some embodiments, the system might further comprise a scale that is used to measure weight of food on a food container during meals consumed by the patient, where the food is consumed out of the food container during the meals; and a user device associated with the user, and communicatively coupled to the scale. The user device might comprise at least one second processor and a second non-transitory computer readable medium communicatively coupled to the at least one second processor. The second non-transitory computer readable medium might have stored thereon computer software comprising a second set of instructions that, when executed by the at least one second processor, causes the user device to: receive the food intake data associated with the patient from the scale; prompt the patient to enter self-reported feelings of satiety from the patient during meals and receive satiety data from the patient; and send food intake and satiety data associated with the patient to the computing system. In some cases, the user device might comprise one of a tablet computer, a smart phone, a mobile phone, a laptop computer, a desktop computer, or a dedicated food intake tracking device, and/or the like.

In some embodiments, the first set of instructions, when executed by the at least one first processor, further causes the computing system to: based on a determination that a likelihood of risk of suicide by the patient is below the first predetermined threshold value but exceeds a second predetermined threshold value, send suggestions to the patient to change eating behavior of the patient toward at least one of eating rates, food amounts, and mealtime durations that correspond to levels designed to stimulate physiological responses that evoke positive feelings for the patient.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### Specific Exemplary Embodiments

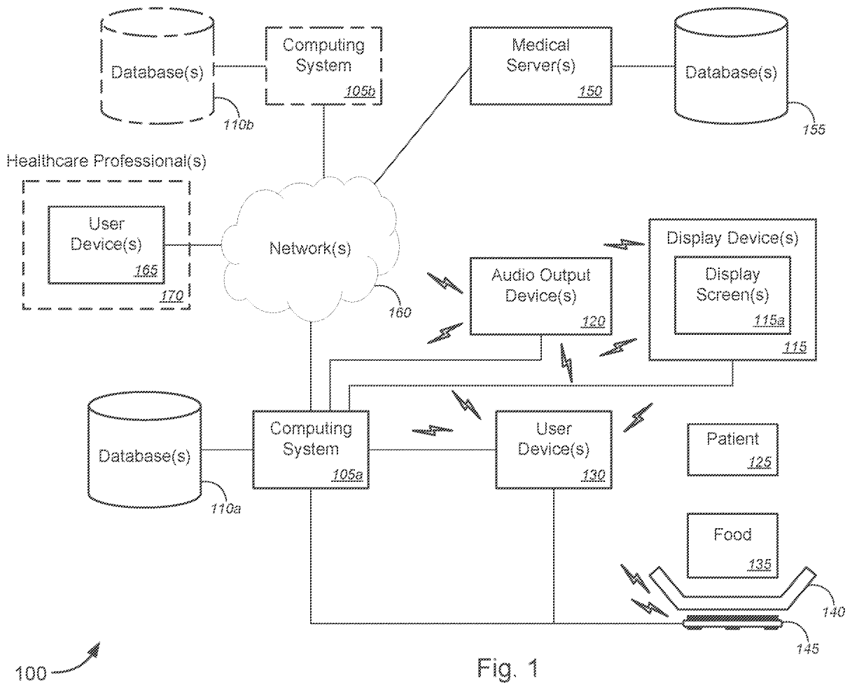

We now turn to the embodiments as illustrated by the drawings. FIGS. 1-6 illustrate some of the features of the method, system, and apparatus for implementing medical or medical-related diagnosis and treatment, and, more particularly, to methods, systems, and apparatuses for implementing risk assessment for suicide and treatment based on interaction with virtual clinician, food intake tracking, and/or satiety determination, as referred to above. The methods, systems, and apparatuses illustrated by FIGS. 1-6 refer to examples of different embodiments that include various components and steps, which can be considered alternatives or which can be used in conjunction with one another in the various embodiments. The description of the illustrated methods, systems, and apparatuses shown in FIGS. 1-6 is provided for purposes of illustration and should not be considered to limit the scope of the different embodiments.

With reference to the figures, FIG. 1 is a schematic diagram illustrating a system 100 for implementing risk assessment for suicide and treatment based on interaction with virtual clinician, food intake tracking, and/or satiety determination, in accordance with various embodiments.

In the non-limiting embodiment of FIG. 1, system 100 might comprise a computing system 105a and a corresponding data store(s) or database(s) 110a that is local to the computing system 105a. In some cases, the database(s) 110a might be external, yet communicatively coupled, to the computing system 105a. In other cases, the database(s) 110a might be integrated within the computing system 105a. System 100, according to some embodiments, might further comprise one or more display devices 115 (collectively, “display devices 115” or “display device(s) 115” or the like), which might each include a display screen 115a. System 100 might further comprise one or more audio output devices 120 (collectively, “audio output devices 120,” “audio output device(s) 120,” or “speakers 120,” or the like) and one or more user devices 130 (collectively, “user devices 120” or “user device(s) 130” or the like), which might each include a touchscreen display or touchscreen display device 120 or other user interface device, and/or the like. System 100 might further comprise a scale 145, which might be a communications-enabled scale that is used to measure weight of food 135 on a food container 140 during meals consumed by a user or patient 125 where the food 135 is consumed out of the food container 140 during the meals. In some cases, the food container 140 might include, but is not limited to, a plate, a bowl, a serving dish, a glass food storage container, a plastic food storage container, a pot, a pan, or other container that is suitable for holding food and for eating out of. For hot containers, like pots, pans, etc., a trivet (not shown) might be used between them and the scale 145. According to some embodiments, the scale 145 might communicate with the user device(s) 130, the computing system 105a, and/or the network 160 via wired communications (e.g., using a universal serial bus (“USB”) cable, or other suitable cable, or the like) and/or via wireless communications (e.g., using at least one of WiFi protocol, Bluetooth™ protocol, Zigbee protocol, Z-wave protocol, or other wireless communications protocols, or the like). In some instances, the scale 145 might be a portable scale that is suitable for fitting within a handbag, a tote bag, a briefcase, a satchel, a backpack, a day pack, or other suitable carrying bag for use while the patient 125 is eating out or at someone else's home (i.e., for times when the patient is not eating at home).

In some cases, the weight as measured on the scale would be zeroed out when the food container 140 is placed on the scale 145 while in the empty state (and also when other objects, such as trivets or the like, are used). In this way, the measurement as indicated on the scale or as recorded and sent by the scale would only reflect the food 135 being placed in the food container 140 as it is being consumed by the patient 125. In alternative cases, zeroing does not occur, and the measurement is of the food 135 and the food container 140 (plus other objects, such as trivets or the like). The scale 145 separately measures and sends the weight of the food container 140 when empty (and any other objects), and the user device(s) 130 (running a software application (“app”) consistent with the various embodiments herein) and/or the computing system 105a might subsequently provide the user with a list of food containers 140 (and other objects used) and their weights. Upon selection of which food container 140 was used (and also which other objects were used with the food container 140 (if at all)) for which meal, the user device(s) 130 and/or the computing system 105a might subsequently subtract the weight of the selected food container 140 (and any other object) from the total weight of the food 135 and the food container 140 (and any other object) for each particular meal. Herein, the weight of the food 135 would be a time-measured weight that reduces over the course or duration of the meal, so as to measure the amount of food 125 as well as the rate of food consumption by the patient 125.

In some embodiments, the computing system 105a (and corresponding database(s) 110a) might be disposed proximate to or near at least one of the display device(s) 115, the audio output device(s) 120, the user device(s) 130, and/or the scale 145, and/or the like. According to some embodiments, alternative or additional to the computing system 105a and corresponding database(s) 110a being disposed proximate to or near the at least one of the display device(s) 115, the audio output device(s) 120, the user device(s) 130, and/or the scale 145, and/or the like, system 100 might comprise remote computing system 105b and corresponding database(s) 110b that communicatively couple with at least one of the display device(s) 115, the audio output device(s) 120, the user device(s) 130, and/or the scale 145, and/or the like, via one or more networks 160. In some cases, the remote computing system 105b might also communicate with computing system 105a via the network(s) 160, in the cases that computing system 105a is used. According to some embodiments, the computing system 105a might include, without limitation, at least one of a tablet computer, a laptop computer, a desktop computer, a local server, a dedicated food intake tracking device, or a user device, and/or the like. In some embodiments, remote computing system 105b might include, but is not limited to, at least one of a server computer over a network, a cloud-based computing system over a network, and/or the like. System 100 might further comprise one or more medical servers 150 and corresponding database(s) 155 that are accessible by at least one of the computing system 105a, the remote computing system 105b, the display device(s) 115, the audio output device(s) 120, the user device(s) 130, and/or the scale 145, and/or the like, via one or more networks 160. System 100 might further comprise, one or more user devices 165 that are associated with corresponding one or more healthcare professionals 170.

In some instances, network(s) 160 might include a local area network (“LAN”), including, without limitation, a fiber network, an Ethernet network, a Token-Ring™ network, and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, the Z-Wave protocol known in the art, the ZigBee protocol or other IEEE 802.15.4 suite of protocols known in the art, and/or any other wireless protocol; and/or any combination of these and/or other networks. In a particular embodiment, the network(s) 160 might include an access network of an Internet service provider (“ISP”). In another embodiment, the network(s) 160 might include a core network of the ISP, and/or the Internet.

Each of at least one of the display device(s) 115, the audio output device(s) 120, the user device(s) 130, and/or the scale 145, and/or the like might communicatively couple (either directly or indirectly) to the computing system 105a, to the network(s) 160, and/or to each other, either via wireless connection (denoted in FIG. 1 by the lightning bolt symbols) and/or via wired connection (denoted in FIG. 1 by the connection lines). The one or more user devices 130 might each receive user input from a patient 125 (in various embodiments, receiving touch input from the patient 125 via a touchscreen display; via typed input from the patient 125 via a physical keyboard, a physical number pad, a physical user interface, and/or a virtual or soft user interface of the touchscreen display; or via voice input from the patient 125 via a microphone(s) or a sound sensor(s), and/or the like), and might each relay the user input to the computing system 105a, the computing system 105b via network(s) 160, and/or the medical server(s) 150 via network(s) 160, according to some embodiments. The one or more display devices 115 might include, but are not limited to, at least one of one or more monitors (e.g., computer monitor or laptop monitor, or the like), one or more television sets (e.g., smart television sets or other television sets, or the like), and/or the like. In some cases, the user device(s) 130 might include, without limitation, one of a laptop computer, a tablet computer, a smart phone, a mobile phone, a personal digital assistant, or a dedicated device used for interacting with a virtual clinician as described herein, and/or the like.

In operation, scale 145 might be used to measure the weight of food 135 on food container 140 during meals consumed by patient 125, with the food 135 being consumed out of the food container 140 during the meals. In this manner, the scale 145 can monitor and track the amount of food being consumed by the patient, while also tracking times of day, the number of meals per day, as well as the rate of food consumption during each meal, and any interruptions or disruptions during meals, and the like (collectively, “food intake data” or the like). The scale 145 might communicatively couple (either via wired or wireless connection), and send the food intake data, to at least one of the user device(s) 130 and/or the computing system 105a, or the like. The user device(s) 130 and/or the computing system 105a might also prompt the patient 125 to enter self-reported feelings of satiety during meals, and might record such self-reported feelings of satiety. The user device(s) 130 might send the food intake and satiety data to the computing system 105a, the computing system 105b, and/or the medical server(s) 150 for analysis, together with data obtained during sessions in which the patient 125 interacts with a virtual clinician, as described below. In some embodiments, the food intake and satiety data might include, without limitation, at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals, and/or the like.

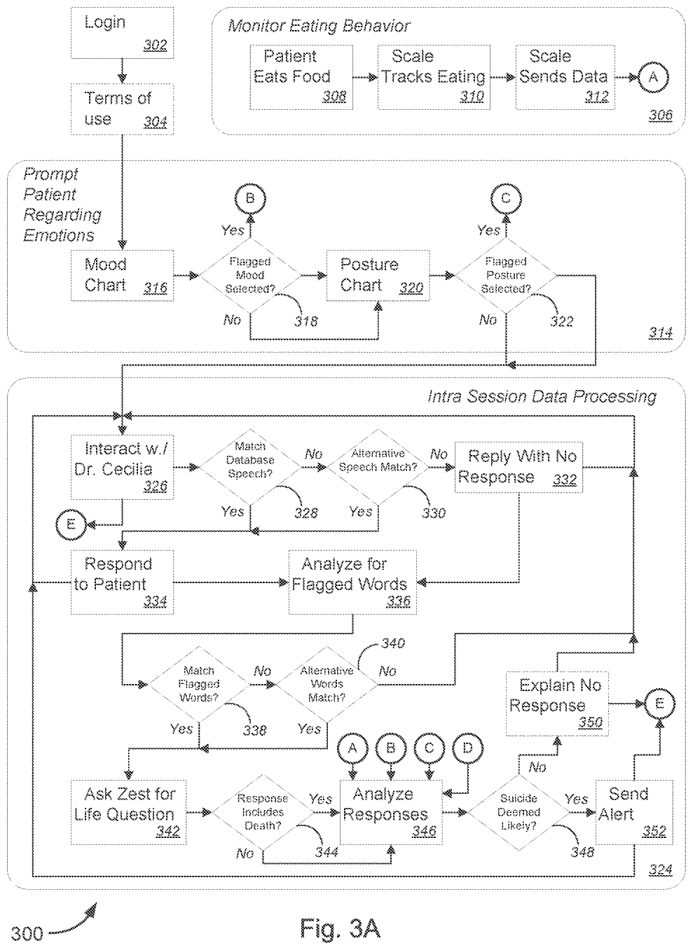

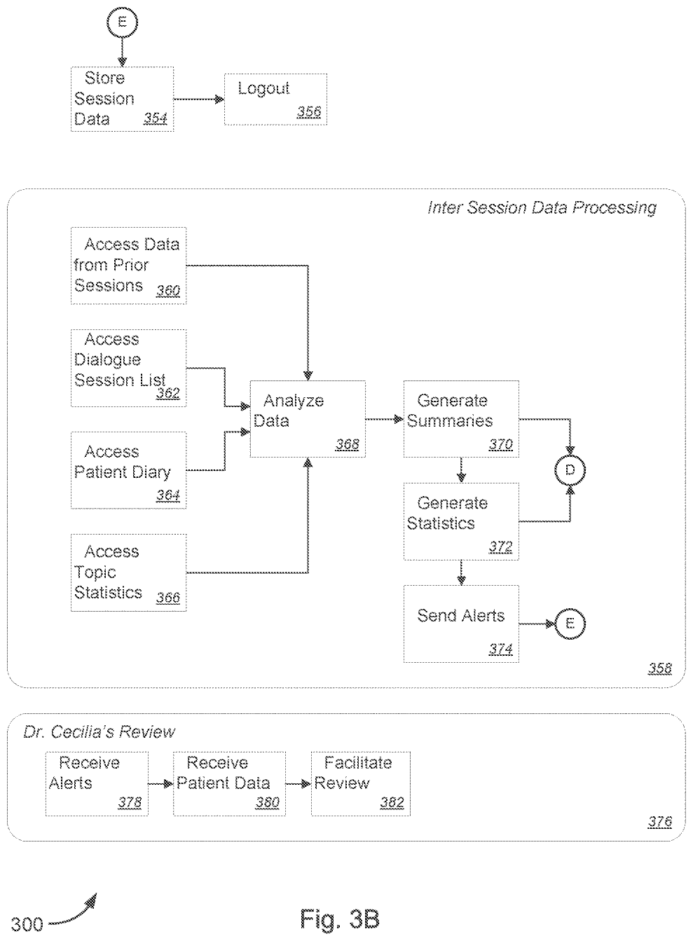

During one or more sessions, the computing system 105a, the computing system 105b, and/or the medical server(s) 150 (collectively, “computing system” or the like) might generate a virtual clinician capable of simulating facial expressions and body expressions (such as the virtual clinician 220, “Dr. Cecilia,” as shown in FIGS. 2B-2E and described with respect to FIGS. 3A and 3B, or the like). The generated virtual clinician would also be capable of interacting with patients to hold conversations, or the like. The computing system might cause the generated virtual clinician to interact with patient 125.

In some embodiments, causing the generated virtual clinician to interact with the patient might comprise causing, with the computing system, the generated virtual clinician to interact with patient 125, via at least one of participating in a conversation with the patient, asking the patient one or more questions, or answering one or more questions posed by the patient, where interactions between the virtual clinician and the patient 125 might be based at least in part on one or more of using, recognizing, or interpreting one or more of words, verbal expressions, statements, sentences, sentence responses, questions, or answers that are stored in a database (e.g., database(s) 110a, 110b, and/or 155, or the like). Alternatively, or additionally, causing the generated virtual clinician to interact with the patient might comprise one of interacting with the patient by displaying the generated virtual clinician on (display screen 115a of) display device 115 and displaying words of the virtual clinician as text on the (display screen 115a of) display device 115; interacting with the patient by displaying the generated virtual clinician on (display screen 115a of) display device 115 and presenting words of the virtual clinician via audio output device 120; or interacting with the patient by displaying the generated virtual clinician on (display screen 115a of) display device 115, presenting words of the virtual clinician via an audio output device 120, and displaying words of the virtual clinician as text on (display screen 115a of) display device 115.

Alternatively, or additionally, causing the generated virtual clinician to interact with the patient might comprise at least one of: (1) prompting the patient, with the computing system, to select a facial expression among a range of facial expressions that represents current emotions of the patient, and receiving, with the computing system, a first response from the patient, the first response comprising a selection of a facial expression that represents current emotions of the patient; (2) prompting the patient, with the computing system, to select a body posture among a range of body postures that represents current emotions of the patient, and receiving, with the computing system, a second response from the patient, the first response comprising a selection of a body posture that represents current emotions of the patient; or (3) prompting the patient, with the computing system, to select a statement regarding zest for life among a range of statements regarding zest for life that represents current thoughts of the patient regarding life and death, and receiving, with the computing system, a third response from the patient, the third response comprising a selection of a statement regarding zest for life that represents current thoughts of the patient regarding life and death; and/or the like.

Alternatively, or additionally, causing the generated virtual clinician to interact with the patient might comprise at least one of: recording video of the patient during the interaction and utilizing at least one of facial analysis, body analysis, or speech analysis to identify at least one of facial expressions of the patient, body language of the patient, or words spoken by the patient; recording audio of the patient during the interaction and utilizing speech analysis to identify words spoken by the patient; or recording words typed by the patient via a user interface device and utilizing text analysis to identify words typed by the patient; and/or the like.

During or after each session, the computing system might identify one or more flagged words or expressions spoken and/or typed by the patient during the interaction. According to some embodiments, identifying one or more flagged words or expressions spoken and/or typed by the patient during the interaction might comprise determining, with the computing system, whether words or expressions spoken or typed by the patient match predetermined flagged words and expressions that are indicative of suicide risk (such as the words and expressions depicted in FIG. 2A, or the like). The computing system might also record, to a datastore (e.g., database(s) 110a, 110b, and/or 155, or the like), the interactions between the virtual clinician and the patient.

While the system can track and analyze interactions and other information regarding the patient 125 during each session (i.e., performing Intra Session Data Processing), the system may also track or analyze across multiple sessions with the patient (i.e., performing Inter Session Data Processing), by compiling and analyzing historical data associated with the patient. Merely by way of example, in some cases, the historical data might include, but is not limited to, at least one of interactions between the virtual clinician and the patient during one or more prior sessions, one or more diary entries entered by the patient, one or more records containing words or expressions previous spoken or typed by the patient that match predetermined flagged words and expression that are indicative of suicide risk, one or more records containing data related to emotions of the patient during prior sessions, one or more prior suicide risk assessments for the patient, or one or more prior medical-related assessments performed on the patient, and/or the like.

In some embodiments, the computing system might analyze patient data to determine likelihood of risk of suicide by the patient. According to some embodiments, the patient data might include, without limitation, at least one of the received food intake and satiety data associated with the patient (as obtained from the scale 145 and/or the user device(s) 130, or the like); the interactions between the virtual clinician and the patient; one or more of the received first response, the received second response, and/or the received third response; or the historical data associated with the patient; and/or the like. Based on a determination that a likelihood of risk of suicide by the patient exceeds a first predetermined threshold value, the computing system might send a message to one or more healthcare professionals 170 (i.e., to one or more user devices 165 associated with corresponding one or more healthcare professional 170) regarding the likelihood of risk of suicide by the patient. Based on a determination that a likelihood of risk of suicide by the patient is below the first predetermined threshold value but exceeds a second predetermined threshold value, the computing system might send suggestions to the patient 125 (e.g., by sending the suggestions to user device(s) 130 associated with patient 125) to change eating behavior of the patient toward at least one of eating rates, food amounts, and mealtime durations that correspond to levels designed to stimulate physiological responses that evoke positive feelings for the patient.

According to some embodiments, analyzing the interactions between the virtual clinician and the patient to determine likelihood of risk of suicide by the patient might comprise utilizing at least one of artificial intelligence functionality or machine learning functionality to determine likelihood of risk of suicide by the patient. In some cases, recording the interactions between the virtual clinician and the patient and analyzing the interactions between the virtual clinician and the patient to determine likelihood of risk of suicide by the patient may be performed in real-time or near-real-time.

In some aspects, the system might be configured to treat the patient 125, by helping patient 125 eat food in a manner that would allow release of satiety hormones in the patient's body to evoke a normal feeling of fullness, by providing audible and/or visual cues prompting the patient to eat either faster or slower if the system determines that the patient is eating too slow or too fast. The system registers eating rate (measured in grams per minute, or the like), amount of food (measured in grams, or the like), and duration of each meal (measured in minutes). A normal meal might consist of 300-350 grams eaten over 12-15 minutes. A healthy subject would display a decelerated eating behavior—that is, eating fast at the beginning of the meal, then slowing near the end of the meal. On the other hand, a subject with an eating disorder or who is considered obese might display a linear eating behavior—that is, eating at the same pace throughout the meal. Subjects with eating disorders or who are considered obese have been found by the inventor to be more likely than healthy subjects to exhibit sad or very sad feelings and to harbor thoughts of suicide. As such, these food intake data are useful as a factor to analyze to determine likelihood of suicide. The system might also prompt the patient at regular intervals to record his, her, or their feelings of fullness (or satiety) (e.g., slight, moderate, strong, very strong, or extreme, or the like). Such satiety data is also useful as another factor to analyze to determine likelihood of suicide.

In another aspect, when interacting with a patient, the virtual clinician (i.e., Dr. Cecilia) might identify words and expressions indicating that the patient does not feel well and is about to harm himself, herself, or themselves (herein, referred to as “flagged words or expressions” or the like). A technique that may be used in identifying flagged words or expressions might include, without limitation, n-gram technique, which utilizes a contiguous sequence of a given number of items or words to identify flagged words or expressions. Examples of (a) a 1-gram (or unigram), (b) a 2-gram (or bigram), (c) a 3-gram (or trigram), or (d) a 4-gram of the expression, “I want to harm myself,” might be as follows: (a) “I,” “want,” “to,” “harm,” and “myself”; (b) “I want,” “want to,” “to harm,” and “harm myself”; (c) “I want to,” want to harm,” and “to harm myself”; and (4) “I want to harm” and “want to harm myself”; and so on. Analysis by using n-grams may facilitate identification of flagged words or expressions.

It has been found by the inventor that patients are more honest when interacting with a virtual clinician, such as Dr. Cecilia, than when interacting with a human clinician, and that computer software is better at the diagnosis of psychological problems than a human physician. When a patient is using a words, expression, or statement indicating that he, she, or they are at risk of suicide, the virtual clinician might ask the patient to select a facial expression that matches his, her, or their emotions, to select a posture that is consistent with that emotion, and to select from a list of statements that reflects the patient's zest for life. From the numerical scale shown in FIG. 2D, for example, if the patient scores 2 or higher on the 0-3 emotion scale, an e-mail, SMS, and/or a telephone signal might alert the physician on duty or other healthcare professional. The conversation or interaction that the patient had with Dr. Cecilia might then be forwarded to the physician, including the frequency of words indicative of risk of suicide over the previous three days.

As discussed herein, the system identifies five measurable concepts that may be tracked, including, for example: (A) flagged words, or expressions (such as shown in the non-limiting example of FIG. 2A); (B) facial expressions (such as shown in the non-limiting example of FIG. 2B); (C) postures (such as shown in the non-limiting example of FIG. 2C); (D) 15 subjects or topics consisting of categorized answers from Dr. Cecilia to questions asked by patients; and (E) eating behavior measured by scale 145 or the like. Regarding (D), the subjects or topics might be classified from “healthy” to “severely ill,” and the answers given by Dr. Cecilia are made to coincide with generation of facial expression on the face of the virtual clinician, Dr. Cecilia, to reflect corresponding concerned, serious, neutral, positive, and/or happy emotions. Regarding (D), the 15 subjects or topics include, but are not limited to, (1) the illness, (2) fear of fatness, (3) striving for thinness, (4) bulimia, (5a) health consequences—psychiatric (e.g., anxiety, compulsion, depression, mood changes, dark thoughts, etc.), (5b) health consequences—physical, (6) social consequences, (7) the treatment (e.g., anxiety reduction, thermal treatment, reward schedule, physical activity, eating behavior and satiety, Mandometer training or training with scale and other tools, food schedule, forbidden foods, etc.), (8) eating behavior and satiety, (9) physical activity, (10) weight, (11) food, (12) social reconstruction, (13) healthcare professionals, (14) remission, and (15) relapse, or the like. Regarding (E), the concept of eating behavior might include three variables: (I) the deviation from a normal meal; (II) eating pattern (whether linear or decelerated eating); and (III) occurrence of displaced behaviors during a meal (e.g., reheating the meal to prolong duration of the meal, which is a typical anorectic behavior).

In terms of treatment of patients, each interaction or session that the patient has with the virtual clinician might be described as a process measuring intensity of concepts and time. The processes of patients in treatment might be compared to the same variables for patients with a successful outcome (i.e., patients in remission or recovery, or the like). The processes might affect each other in time series. By dichotomizing the individual responses into sets of patients and non-patients, one can generate a graphs of the risk behavior for healthy vs. unhealthy individuals. For example, a successful treatment of a patient might show the number or intensity of flagged words or expressions starting high, then decreasing over the course of days, weeks, or months. Whereas, an unsuccessful treatment of a patient might show the number or intensity of flagged words or expressions remaining substantially unchanged over the course of days, weeks, or months. In some embodiments, treatment of the patient 125 might include, without limitation, interactions with the patient that promote more decelerated eating behaviors, and/or interactions with the patient that aim to discover the source(s) of sadness or depression in the patient and suggesting ways to address or overcome these underlying issues (e.g., anxiety, compulsion, depression, mood changes, dark thoughts, social interaction issues, food related issues, weight or body-image related issues, etc.), and/or interactions with the patient that aim to discover positive aspects of the patient's life and suggesting ways for the patient to focus on those positive aspects, and/or the like.

These and other functions of the system 100 (and its components) are described in greater detail below with respect to FIGS. 2-4.

FIGS. 2A-2E (collectively, “FIG. 2”) illustrate non-limiting examples 200 of parameters that are used to implement risk assessment for suicide and treatment, in accordance with various embodiments. In particular, FIG. 2A is a schematic diagram illustrating a non-limiting example 200 of flagged words and expressions that are stored in a datastore and that may be used to implement risk assessment for suicide and treatment, in accordance with various embodiments. FIGS. 2B-2E are schematic diagrams illustrating a non-limiting example 200 of various interfaces that may be used for implementing risk assessment for suicide and treatment based on interaction with virtual clinician, food intake tracking, and/or satiety determination may be implemented, in accordance with various embodiments.

Referring to FIG. 2A, a list of flagged words and expressions 205 might be stored, and accessed from, database(s) 210, which might correspond to one or more of database(s) 110a, 110b, and/or 155 of FIG. 1, or the like. The list of words and expressions 205 that are listed in FIG. 2A, some with particular emphasis on certain words in certain expressions represent words and expressions that are known to the inventor for being associated with suicidal ideation of patients. Although a specific number of words and expressions are depicted in FIG. 2A (with 85 expressions being listed), the various embodiments are not so limited, and the listed words and expressions are merely illustrative of words and expressions that are likely to be indicative of suicidal thoughts by patients, and other similar words and expressions may also be used as flagged words and expressions for triggering further analysis to determine likelihood of a patient to commit or attempt suicide.

In FIGS. 2B-2E, a user interface 215 is depicted, in which a virtual clinician 220 (in this case, “Dr. Cecilia”) is generated and presented (on a display screen of a display device, such as display screen 115a of display device(s) 115 of FIG. 1, or the like) as a virtual construct that is capable of simulating facial expressions and body expressions, and, of course, capable of interaction with patients to hold conversations, or the like. The virtual clinician 220 is presented within a virtual environment 225 (in this case, an environment simulating a counselor's or therapist's office or consultation room), which in various embodiments would be configured to simulate a safe, calm, relaxing, and/or receptive environment to encourage patients to openly and willingly interact with and respond to the virtual clinician 220.

As shown in FIGS. 2B-2E, the user interface 215 might display written words 235a-235d (collectively, “written words 235” or the like) of the virtual clinician 220, in some cases, concurrent with the generated virtual clinician being depicted as saying the words (not shown), and, in other cases, also concurrent with an audio output device (e.g., audio output device(s) 120 of FIG. 1, or the like) presenting an aural or simulated spoken version of the words. The user interface 215 might also provide text input fields 240a-240d (collectively, “text input fields 240” or the like) that enable the patient to manually type in sentences, words, responses, and/or questions. In some cases, where a microphone or other sound sensor is available and is linked to the system, the patient might speak sentences, words, responses, and/or questions into the microphone or other sound sensor, and the system might record, convert, and attempt to match the sentences, words, responses, and/or questions with sentences, words, responses, and/or questions stored in a database(s), and, in some cases, might display a speech-to-text conversion of the patient's sentences, words, responses, and/or questions in the text input field 240. The user interface 215 might further provide the patient with various options, including, but not limited to, an option to end the conversation 245a, an option to access the patient's diary 245b, and/or the like.

With reference to FIG. 2B, the user interface 215 might display a range of faces 230 having facial expressions representing varying degrees of emotions, ranging from very sad 230a, sad 230b, neutral 230c, glad 230d, and happy 230e, or the like. Although five faces 230 with facial expressions representing emotions are depicted in FIG. 2B, the various embodiments are not so limited, and any suitable number of faces 230 with facial expressions representing emotions may be presented as appropriate or as desired. However, too few faces with facial expressions representing emotions might miss emotions felt by the patient, while too many faces with facial expressions representing emotions might confuse the patient. Here, because the goal is to identify potential suicidal thoughts or tendencies in patients, the emotions are selected to correspond to emotions that are likely to result in suicidal thoughts or tendencies in patients, along with the opposite emotions and a neutral expression, to present a spectrum of emotions.

As shown in FIG. 2B, the user interface 215 might display written words 235a of the virtual clinician 220, in some cases, concurrent with the generated virtual clinician being depicted as saying the words, and, in other cases, also concurrent with an audio output device presenting an aural or simulated spoken version of the words. In this case, the written words 235a might include, without limitation, “Before we continue: please select the expression that best matches how you feel right now” [herein, referred to as “a mood chart question”]. The patient would then respond by selecting one of the faces 230 having a facial expression that represents current emotions of the patient, by clicking, tapping, or highlighting one of the faces 230. The patient may also enter his, her, or their thoughts in the text input field 240a, either by typing and/or speaking (which is converted into text and subsequently auto-filled) words or expressions (in this case, “I want to end this” as depicted in the example of FIG. 2B). The patient's interactions with the virtual clinician as well as the patient's response to the mood chart question would subsequently or concurrently be recorded to a database(s) (e.g., database(s) 110a, 110b, and/or 155, or the like) and analyzed by a computing system (e.g., at least one of computing system 105a, computing system 105b, user device(s) 130, and/or medical server(s) 150 of FIG. 1, or the like), in some cases, in conjunction with artificial intelligence (“AI”) and/or machine learning systems, to determine likelihood of risk of suicide by the patient, and also to track progress of the patient during treatments to address, mitigate, minimize, and/or divert suicidal thoughts and feelings.

Turning to FIG. 2C, similar to FIG. 2B, the user interface 215 might display a range of body postures 250 that represent varying degrees of emotions, ranging from very sad 250a, sad 250b, neutral 250c, glad 250d, and happy 250e, or the like. Although five body postures 250 that represent emotions are depicted in FIG. 2C, the various embodiments are not so limited, and any suitable number of body postures 250 that represent emotions may be presented as appropriate or as desired. However, too few body postures that represent emotions might miss emotions felt by the patient, while too many body postures that represent emotions might confuse the patient. Here, because the goal is to identify potential suicidal thoughts or tendencies in patients, the emotions are selected to correspond to emotions that are likely to result in suicidal thoughts or tendencies in patients, along with the opposite emotions and a neutral expression, to present a spectrum of emotions.

As shown in FIG. 2C, the user interface 215 might display written words 235b of the virtual clinician 220, in some cases, concurrent with the generated virtual clinician being depicted as saying the words, and, in other cases, also concurrent with an audio output device presenting an aural or simulated spoken version of the words. In this case, the written words 235a might include, without limitation, “Before we continue: please select the posture that best matches how you feel right now” [herein, referred to as “a posture question”]. The patient would then respond by selecting one of the body postures 250 having a facial expression that represents current emotions of the patient, by clicking, tapping, or highlighting one of the body postures 250. The patient may also enter his, her, or their thoughts in the text input field 240b, either by typing and/or speaking (which is converted into text and subsequently auto-filled) words or expressions (in this case, “I want to end this” as depicted in the example of FIG. 2C). The patient's interactions with the virtual clinician as well as the patient's response to the posture question would subsequently or concurrently be recorded to a database(s) (e.g., database(s) 110a, 110b, and/or 155, or the like) and analyzed by a computing system (e.g., at least one of computing system 105a, computing system 105b, user device(s) 130, and/or medical server(s) 150 of FIG. 1, or the like), in some cases, in conjunction with the use of artificial intelligence (“AI”) and/or machine learning systems and in conjunction with analysis of the patient's response to the mood chart question depicted in FIG. 2B (if available), to determine likelihood of risk of suicide by the patient, and also to track progress of the patient during treatments to address, mitigate, minimize, and/or divert suicidal thoughts and feelings.

Referring to FIG. 2D, if the patient responded to the mood chart question and/or the posture question by selecting a face having facial expression and/or by selecting a posture that represents an emotion that is flagged for being likely indicative of suicidal thoughts or tendencies (i.e., an emotion including, but not limited to, sad or very sad, or the like), the user interface 215 might display a question regarding the patient's zest for life and a prompt to select a statement regarding zest for life among a range of statements regarding zest for life that represents current thoughts of the patient regarding life and death, in some cases, with an arbitrary numerical scale. For example, as shown in FIG. 2D, the user interface 215 might display written words 235c of the virtual clinician 220, in some cases, concurrent with the generated virtual clinician being depicted as saying the words, and, in other cases, also concurrent with an audio output device presenting an aural or simulated spoken version of the words. In this case, the written words 235a might include, without limitation, “I also have a question for you. This question concerns your appetite for life, and whether you have felt listless and weary of life. Have you had thoughts of suicide, and if so to what extent do you consider it a realistic escape? On the scale below, please select which answer you think best indicates your condition during the past three days” [herein, referred to as “a zest for life question”]. The numerical scale and corresponding statements regarding zest for life 255 might include, without limitation: (i) “0—My appetite for life is normal” 255a; (ii) “0.5” 255b; (iii) “1—Life doesn't seem particularly meaningful, though I don't wish I were dead” 255c; (iv) “1.5” 255d; (v) “2—I often think it would be better to be dead, and though I don't really want to commit suicide it does seem a possible solution” 255e; (vi) “2.5” 255f; and (vii) “3—I am quite convinced that my only solution is to die and I give a lot of thought to the best way to take my own life” 255g; and/or the like. Although seven options or statements regarding zest for life 255a-255g are depicted in FIG. 2D, the various embodiments are not so limited, and any suitable number of options or statements regarding zest for life 255 may be presented as appropriate or as desired.

The patient would then respond by selecting one of the options or statements regarding zest for life 255, by clicking, tapping, or highlighting one of the options or statements regarding zest for life 255. The patient may also enter his, her, or their thoughts in the text input field 240c, either by typing and/or speaking (which is converted into text and subsequently auto-filled) words or expressions (in this case, “I want to end this” as depicted in the example of FIG. 2D). The patient's interactions with the virtual clinician as well as the patient's response to the zest for life question would subsequently or concurrently be recorded to a database(s) (e.g., database(s) 110a, 110b, and/or 155, or the like) and analyzed by a computing system (e.g., at least one of computing system 105a, computing system 105b, user device(s) 130, and/or medical server(s) 150 of FIG. 1, or the like), in some cases, in conjunction with the use of artificial intelligence (“AI”) and/or machine learning systems and in conjunction with analysis of the patient's response to the mood chart question depicted in FIG. 2B (if available) and/or analysis of the patient's response to the posture question depicted in FIG. 2C (if available), to determine likelihood of risk of suicide by the patient, and also to track progress of the patient during treatments to address, mitigate, minimize, and/or divert suicidal thoughts and feelings. At any time, the patient may end the conversation by selecting the end conversation option 245a or may access the patient's diary by selecting the diary option 245b. In some embodiments, if the patient scores 2 or higher on the 0-3 emotion scale, an e-mail, SMS, and/or a telephone signal might alert the physician on duty or other healthcare professional. Although FIG. 2D depicts a 0-3 emotion scale, the various embodiments are not so limited, and any suitable numerical scale may be used; in some cases, an alphabetic scale may be used, while in other cases, only statements might be listed and only selection of statements containing references to or indications that the patient is thinking about death as an option would trigger contacting the physician on duty or other healthcare professional (via e-mail, SMS, telephone, etc.). The conversation or interaction that the patient had with Dr. Cecilia might then be forwarded to the physician, including the frequency of words indicative of risk of suicide over the previous three days

With reference to FIG. 2E, in the event that the computing system determines that a likelihood of risk of suicide by the patient exceeds a first predetermined threshold value or exceeds some pre-established threshold level, the computing system might send an alert message to one or more healthcare professionals regarding the likelihood of risk of suicide by the patient, and might cause the user interface 215 to display written words 235d of the virtual clinician 220, in some cases, concurrent with the generated virtual clinician being depicted as saying the words, and, in other cases, also concurrent with an audio output device presenting an aural or simulated spoken version of the words. In this case, the written words 235a might include, without limitation, “Your answer concerns me and I will now inform the staff. You can of course also contact the Mandometer clinic yourself: 08-556 406 53. You can now ask a new question” [herein, referred to as “an alert notification”]. The patient may then ask a new question in the text field 240d. At any time, the patient may end the conversation by selecting the end conversation option 245a or may access the patient's diary by selecting the diary option 245b.

These and other functionalities of the system (and its components) are described in greater detail above with respect to FIGS. 1, 3, and 4.

FIGS. 3A and 3B (collectively, “FIG. 3”) are schematic block flow diagrams illustrating a method 300 for implementing risk assessment for suicide and treatment based on interaction with virtual clinician, food intake tracking, and/or satiety determination, in accordance with various embodiments.

With reference to FIG. 3A, a patient might login to the system (at block 302). For first time patients, the patient might have to read through and accept terms of use (at block 304). The system might monitor eating behavior of the patient (at block 306), which might include the patient eating food during meals (at block 308), a scale (e.g., scale 145 of FIG. 1, or the like) tracking food consumption by the patient (at block 310), and the scale sending the food consumption data (at block 312). The system might send the food consumption data for analysis at block 346 following the circular marker denoted, “A.” After the patient logs in to the system, the system might prompt the patient regarding the patient's current emotions (at block 314), which might include presenting a mood chart and prompting the patient with a mood chart question (at block 316; similar to the mood chart question depicted in FIG. 2B, or the like), determining whether a flagged mood has been selected (at block 318), presenting a posture chart and prompting the patient with a posture question (at block 320; similar to the posture question depicted in FIG. 2C, or the like), and determining whether a flagged posture has been selected (at block 322). If a flagged mood has been selected (at block 318), the system might send the flagged mood for analysis at block 346 following the circular marker denoted, “B.” If not, and in any event, the system might continue to the posture chart and question (at block 320). If a flagged posture has been selected (at block 322), the system might send the flagged posture for analysis at block 346 following the circular marker denoted, “C.” If not, and in any event, the system might continue to Intra Session Data Processing (at block 324).

During Intra Session Data Processing, the patient might interact with Dr. Cecilia (at block 326), which is a virtual clinician or a virtual construct that is capable of simulating facial expressions and body expressions, and, of course, capable of interaction with patients to hold conversations, or the like. The system might determine whether the patient's sentences, words, responses, and/or questions match the sentences, words, responses, and/or questions that are stored in a database(s) (e.g., database(s) 110a, 110b, and/or 155 of FIG. 1, or the like) (at block 328). In particular, the database(s) might store questions and corresponding responses and/or statements and corresponding responses covering the 15 subjects or topics described above with respect to FIG. 1, or the like. If so, the system might proceed to block 334. If not, the system might determine whether the patient's sentences, words, responses, and/or questions match alternative sentences, words, responses, and/or questions that are stored in a database(s) (e.g., database(s) 110a, 110b, and/or 155 of FIG. 1, or the like) (at block 330). In such cases, the database(s) might store alternative sentences, words, responses, and/or questions related to or corresponding to the 15 subjects or topics described above. If so, the system might proceed to block 334. If not, the system would reply to the patient with a no response statement (e.g., “Sorry, I do not understand”; “Please rephrase”; or the like) (at block 332). The system might then loop back to block 326. At block 334, the system might respond to the patient with a response accessed from its database(s). The system might then loop back to block 326.

Concurrently, or subsequently, any and all sentences, words, responses, and/or questions inputted by the patient might be analyzed for any flagged words or expressions (at block 336). The system might determine whether the sentences, words, responses, and/or questions inputted by the patient match any flagged words or expressions (e.g., the flagged words or expressions depicted in FIG. 2A, or the like). If so, the system might proceed to block 342. If not, the system might determine whether the sentences, words, responses, and/or questions inputted by the patient match any alternative words or expressions (e.g., words that are alternative to the flagged words or expressions depicted in FIG. 2A, or the like). If so, the system might proceed to block 342. If not, the system might loop back to block 326. At block 342, the system might ask the patient the zest for life question and might prompt the patient to select a statement regarding zest for life among a range of statements regarding zest for life that represents current thoughts of the patient regarding life and death (e.g., as depicted in FIG. 2D, or the like). The system might then determine whether the patient's response includes mention of death (at block 344). Regardless of the outcome of such determination, the data is sent for analysis at block 346. At block 346, the system might analyze at least one of the food consumption data from block 312 (following circular marker denoted, “A”), the flagged mood data from block 318 (following circular marker denoted, “B”), the flagged posture data from block 322 (following circular marker denoted, “C”), the historical data (if any) from subsequent blocks 370 and/or 372 (following circular marker denoted, “D”), or the patient's response to the zest for life question from block 344, and/or the like. The system might determine a likelihood of risk of suicide by the patient (at block 348). If not, the system might provide an explanation for a no response in light of at least the flagged words and expressions being tracked at block 338 or 340 (at block 350; optional). If so, the system might send an alert message to one or more healthcare professionals regarding the likelihood of risk of suicide by the patient (at block 352). The system might then either loop back to block 326 or might proceed to block 354 in FIG. 3B, following circular marker denoted, “E.” At block 354, the system might store session data, and might proceed to logout (at block 356). At any time during the patient's interaction with Dr. Cecilia, the patient is provided with an option to logout, which is depicted in FIG. 3A by following the circular marker denoted, “E,” extending from block 326.

Referring to FIG. 3B, session data stored in multiple sessions (at block 354) might enable Inter Session Data Processing (at block 358), which might include at least one of accessing data from prior sessions (at block 360), accessing dialogue session list data (at block 362), accessing patient diary data (at block 364), or accessing topic statistics (at block 366), and/or the like. At block 368, the system might then analyze the data from the at least one of block 360, block 362, block 364, and/or block 366, or the like. The system might generate summaries (at block 370) and/or might generate statistics (at block 372) (collectively, “historical data” or the like), based on such analysis, and might send such historical data for analysis at block 346 following the circular marker denoted, “D.” The system might then send alerts or alert messages to one or more healthcare professionals regarding the historical data associated with the patient (at block 374). The system might then proceed to logout by proceeding to block 354 following circular marker denoted, “E.”

The system might also provide the healthcare professionals with options to review the data regarding each patient, as depicted by “Dr. Cecilia's Review” (at block 376), which might include, but is not limited to, receiving the alerts or alert messages (e.g., from blocks 352 and/or 374, or the like) (at block 378), receiving patient data (e.g., from blocks 312, 318, 322, 344, 354, 370, 372, and/or the like) (at block 380), and providing functionalities or features to facilitate review (including, but not limited to, generating summaries (e.g., at block 370), generating statistics (e.g., at block 372), generating diagrams (not shown), generating flow charts, generating reports, and/or the like) (at block 382).

These and other functionalities of the system (and its components) are described in greater detail above with respect to FIGS. 1, 2, and 4.

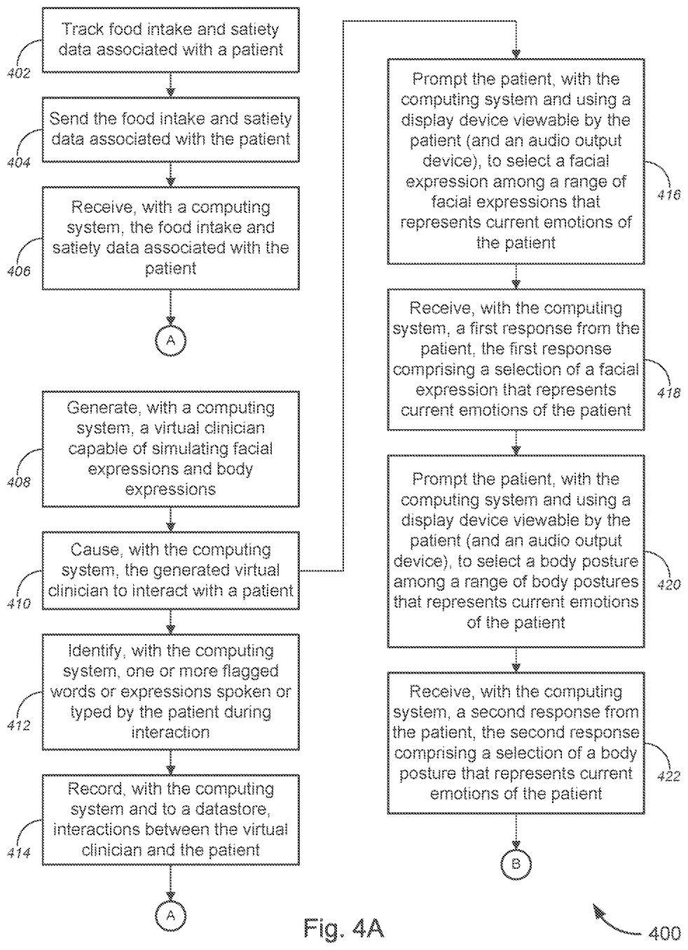

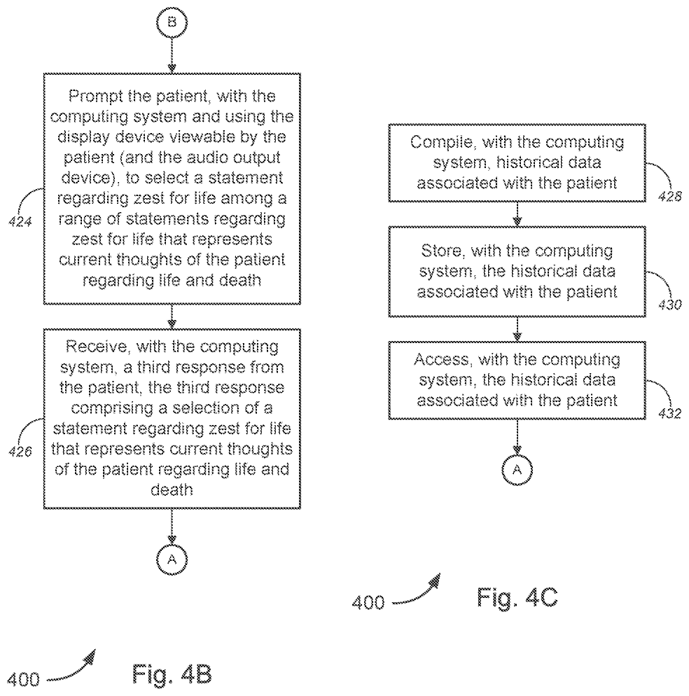

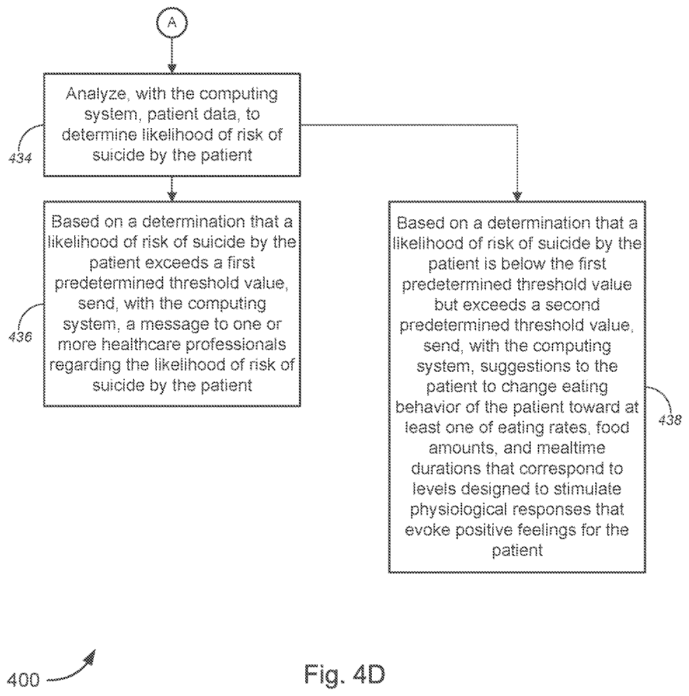

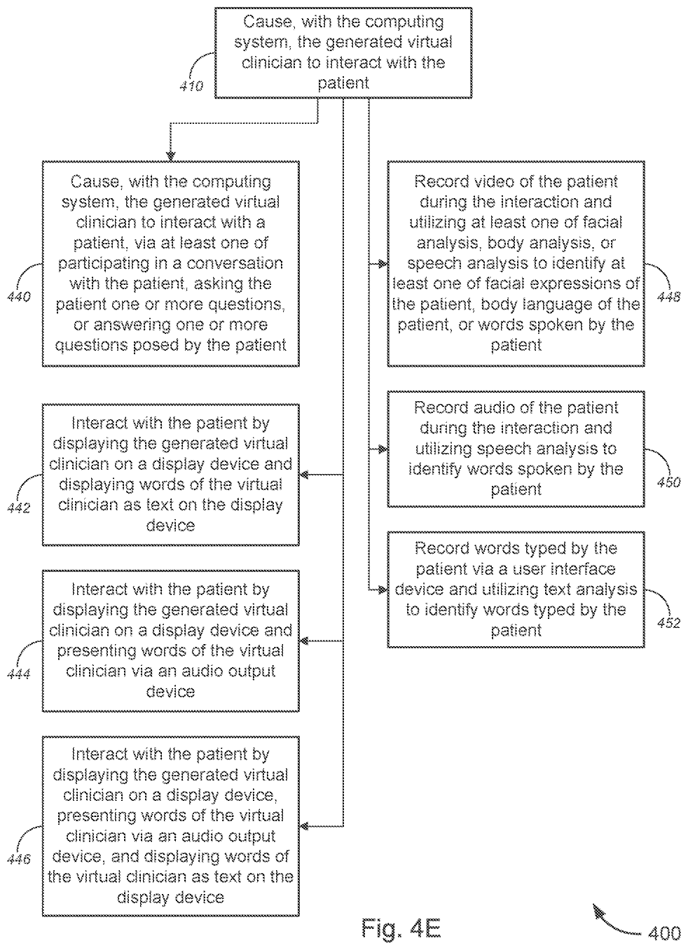

FIGS. 4A-4E (collectively, “FIG. 4”) are flow diagrams illustrating a method 400 for implementing risk assessment for suicide and treatment based on interaction with virtual clinician, food intake tracking, and/or satiety determination, in accordance with various embodiments. Method 400 of FIGS. 4A, 4B, and 4C continues onto FIG. 4D following the circular marker denoted, “A,” while method 400 of FIG. 4A continues onto FIG. 4B following the circular marker denoted, “B.”

While the techniques and procedures are depicted and/or described in a certain order for purposes of illustration, it should be appreciated that certain procedures may be reordered and/or omitted within the scope of various embodiments. Moreover, while the method 400 illustrated by FIG. 4 can be implemented by or with (and, in some cases, are described below with respect to) the systems, examples, or embodiments 100, 200, and 300 of FIGS. 1, 2, and 3, respectively (or components thereof), such methods may also be implemented using any suitable hardware (or software) implementation. Similarly, while each of the systems, examples, or embodiments 100, 200, and 300 of FIGS. 1, 2, and 3, respectively (or components thereof), can operate according to the method 400 illustrated by FIG. 4 (e.g., by executing instructions embodied on a computer readable medium), the systems, examples, or embodiments 100, 200, and 300 of FIGS. 1, 2, and 3 can each also operate according to other modes of operation and/or perform other suitable procedures.

In the non-limiting embodiment of FIG. 4A, method 400, at block 402, might comprise tracking food intake and satiety data associated with a patient. In some embodiments, tracking food intake and satiety data associated with the patient might be performed using at least one of a communications-enabled scale that is used to measure weight of food on a food container during meals consumed by the patient where the food is consumed out of the food container during the meals, a user device that is communicatively coupled to the communications-enabled scale, or the user device that records self-reported feelings of satiety from the patient during meals, and/or the like. According to some embodiments, the food intake and satiety data might include, without limitation, at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals, and/or the like.

At block 404, method 400 might comprise sending the food intake and satiety data associated with the patient. In some cases, the food intake and satiety data might be sent to at least one of a computing system (whether a computing system that is local to the patient or the scale measuring food intake during meals, or a computing system or server that is remote and accessible via a network, or the like) or a user device associated with the patient. Method 400 might further comprise, at block 406, receiving, with the computing system (either directly as a result of the process at block 404, or indirectly via the user device, or both), the food intake and satiety data associated with the patient. Method 400 might continue onto the process at block 434 in FIG. 4D following the circular marker denoted, “A.”

Method 400 might further comprise generating, with a computing system (which may be the same computing system as described above with respect to the processes at blocks 404 and 406, or a different computing system, or the like), a virtual clinician capable of simulating facial expressions and body expressions (block 408). Method 400, at block 410, might comprise causing, with the computing system, the generated virtual clinician to interact with a patient. Method 400 might continue onto the process at block 412 and/or might continue onto the process at block 416.

At block 412, method 400 might comprise identifying, with the computing system, one or more flagged words or expressions spoken and/or typed by the patient during the interaction. According to some embodiments, identifying one or more flagged words or expressions spoken and/or typed by the patient during the interaction might comprise determining, with the computing system, whether words or expressions spoken or typed by the patient match predetermined flagged words and expressions that are indicative of suicide risk (such as the words and expressions depicted in FIG. 2A, or the like). Method 400 might further comprise, at block 414, recording, with the computing system and to a datastore, interactions between the virtual clinician and the patient. Method 400 might continue onto the process at block 434 in FIG. 4D following the circular marker denoted, “A.”

Alternatively, or additionally, method 400 might further comprise prompting the patient, with the computing system, to select a facial expression among a range of facial expressions that represents current emotions of the patient (block 416), and receiving, with the computing system, a first response from the patient, the first response comprising a selection of a facial expression that represents current emotions of the patient (block 418). Alternatively, or additionally, method 400 might further comprise prompting the patient, with the computing system, to select a body posture among a range of body postures that represents current emotions of the patient (block 420), and receiving, with the computing system, a second response from the patient, the first response comprising a selection of a body posture that represents current emotions of the patient (block 422). Alternatively, or additionally, method 400 might continue onto the process at block 424 in FIG. 4B following the circular marker denoted, “B,” where method 400 might further comprise prompting the patient, with the computing system, to select a statement regarding zest for life among a range of statements regarding zest for life that represents current thoughts of the patient regarding life and death (block 424), and receiving, with the computing system, a third response from the patient, the third response comprising a selection of a statement regarding zest for life that represents current thoughts of the patient regarding life and death (block 426). Method 400 might continue onto the process at block 434 in FIG. 4D following the circular marker denoted, “A.”

Merely by way of example, in some cases, prompting the patient (at blocks 416, 420, and 424) may take the form of displaying the prompts as text questions and/or displayed graphics or diagrams (such as shown in FIGS. 2B, 2C, and 2D, or the like) on a display screen of a display device (e.g., a monitor, a television, a display screen of a smart phone, a display screen of a tablet computer, a display screen of a laptop computer, etc.) and/or may take the form of audio prompts by the system using an audio output device (e.g., a speaker(s), or the like). Receiving the first, second, and third responses (at blocks 418, 422, and 426) may take the form of receiving typed input from the patient using at least one of a keyboard, a number pad, a touchscreen display, or other user interface device, or the like, and/or receiving spoken input form the patient using a microphone or the like.

With reference to FIG. 4C, method 400 might further comprise compiling, with the computing system, historical data associated with the patient (block 428), and storing, with the computing system, the historical data associated with the patient (block 430). In some embodiments, the historical data might include, but is not limited to, at least one of interactions between the virtual clinician and the patient during one or more prior sessions, one or more diary entries entered by the patient, one or more records containing words or expressions previous spoken or typed by the patient that match predetermined flagged words and expression that are indicative of suicide risk, one or more records containing data related to emotions of the patient during prior sessions, one or more prior suicide risk assessments for the patient, or one or more prior medical-related assessments performed on the patient, and/or the like. At block 432, method 400 might comprise accessing, with the computing system, the historical data associated with the patient. Method 400 might continue onto the process at block 434 in FIG. 4D following the circular marker denoted, “A.”

At block 434 in FIG. 4D (following the circular marker denoted, “A” from block 406 of FIG. 4A, from block 414 of FIG. 4A, from block 426 of FIG. 4B, and/or from block 432 of FIG. 4C), method 400 might comprise analyzing, with the computing system, patient data to determine likelihood of risk of suicide by the patient. In some embodiments, the patient data might include, without limitation, at least one of the received food intake and satiety data associated with the patient (from block 406 of FIG. 4A), the interactions between the virtual clinician and the patient (from block 414 of FIG. 4A), one or more of the received first response, the received second response, and/or the received third response (from block 426 of FIG. 4B), or the historical data associated with the patient (from block 432 of FIG. 4C), and/or the like. Method 400 might further comprise, based on a determination that a likelihood of risk of suicide by the patient exceeds a first predetermined threshold value, sending, with the computing system, a message to one or more healthcare professionals regarding the likelihood of risk of suicide by the patient (block 436). Alternatively, or additionally, method 400 might further comprise, at block 438, based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.

Referring to FIG. 4E, causing the generated virtual clinician to interact with the patient (at block 410) might comprise causing, with the computing system, the generated virtual clinician to interact with a patient, via at least one of participating in a conversation with the patient, asking the patient one or more questions, or answering one or more questions posed by the patient, and/or the like (block 440). In some instances, interactions between the virtual clinician and the patient may be based at least in part on one or more of using, recognizing, or interpreting one or more of words, verbal expressions, statements, sentences, sentence responses, questions, or answers that are stored in a database, and/or the like.

Alternatively, or additionally, causing the generated virtual clinician to interact with the patient (at block 410) might comprise one of: interacting with the patient by displaying the generated virtual clinician on a display device and displaying words of the virtual clinician as text on the display device (block 442); interacting with the patient by displaying the generated virtual clinician on a display device and presenting words of the virtual clinician via an audio output device (block 444); or interacting with the patient by displaying the generated virtual clinician on a display device, presenting words of the virtual clinician via an audio output device, and displaying words of the virtual clinician as text on the display device (block 446); or the like.

Alternatively, or additionally, causing the generated virtual clinician to interact with the patient (at block 410) might comprise at least one of: recording video of the patient during the interaction and utilizing at least one of facial analysis, body analysis, or speech analysis to identify at least one of facial expressions of the patient, body language of the patient, or words spoken by the patient (block 448); recording audio of the patient during the interaction and utilizing speech analysis to identify words spoken by the patient (block 450); or recording words typed by the patient via a user interface device and utilizing text analysis to identify words typed by the patient (block 452); and/or the like.

In some embodiments, recording the interactions between the virtual clinician and the patient (at block 414) and analyzing the interactions between the virtual clinician and the patient to determine likelihood of risk of suicide by the patient (at block 434) might be performed in real-time or near-real-time. According to some embodiments, analyzing the interactions between the virtual clinician and the patient to determine likelihood of risk of suicide by the patient (at block 434) might be performed utilizing at least one of artificial intelligence functionality or machine learning functionality, and/or the like.

Exemplary System and Hardware Implementation

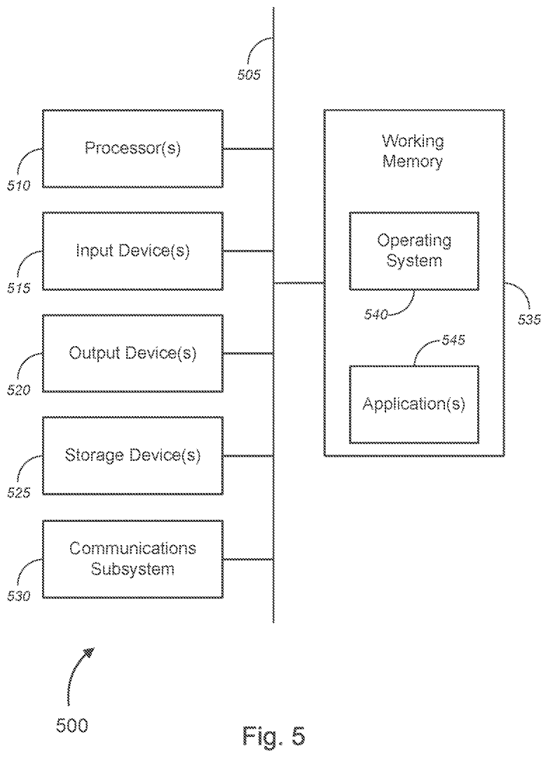

FIG. 5 is a block diagram illustrating an exemplary computer or system hardware architecture, in accordance with various embodiments. FIG. 5 provides a schematic illustration of one embodiment of a computer system 500 of the service provider system hardware that can perform the methods provided by various other embodiments, as described herein, and/or can perform the functions of computer or hardware system (i.e., computing systems 105a and 105b, display device(s) 115, audio output device(s) 120, user device(s) 130 and 165, scale 145, and medical server(s) 150, etc.), as described above. It should be noted that FIG. 5 is meant only to provide a generalized illustration of various components, of which one or more (or none) of each may be utilized as appropriate. FIG. 5, therefore, broadly illustrates how individual system elements may be implemented in a relatively separated or relatively more integrated manner.

The computer or hardware system 500—which might represent an embodiment of the computer or hardware system (i.e., computing systems 105a and 105b, display device(s) 115, audio output device(s) 120, user device(s) 130 and 165, scale 145, and medical server(s) 150, etc.), described above with respect to FIGS. 1-4—is shown comprising hardware elements that can be electrically coupled via a bus 505 (or may otherwise be in communication, as appropriate). The hardware elements may include one or more processors 510, including, without limitation, one or more general-purpose processors and/or one or more special-purpose processors (such as microprocessors, digital signal processing chips, graphics acceleration processors, and/or the like); one or more input devices 515, which can include, without limitation, a mouse, a keyboard, and/or the like; and one or more output devices 520, which can include, without limitation, a display device, a printer, and/or the like.

The computer or hardware system 500 may further include (and/or be in communication with) one or more storage devices 525, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer or hardware system 500 might also include a communications subsystem 530, which can include, without limitation, a modem, a network card (wireless or wired), an infra-red communication device, a wireless communication device and/or chipset (such as a Bluetooth™ device, an 802.11 device, a WiFi device, a WiMax device, a WWAN device, cellular communication facilities, etc.), and/or the like. The communications subsystem 530 may permit data to be exchanged with a network (such as the network described below, to name one example), with other computer or hardware systems, and/or with any other devices described herein. In many embodiments, the computer or hardware system 500 will further comprise a working memory 535, which can include a RAM or ROM device, as described above.

The computer or hardware system 500 also may comprise software elements, shown as being currently located within the working memory 535, including an operating system 540, device drivers, executable libraries, and/or other code, such as one or more application programs 545, which may comprise computer programs provided by various embodiments (including, without limitation, hypervisors, VMs, and the like), and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be encoded and/or stored on a non-transitory computer readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and/or provided in an installation package, such that the storage medium can be used to program, configure, and/or adapt a general purpose computer with the instructions/code stored thereon. These instructions might take the form of executable code, which is executable by the computer or hardware system 500 and/or might take the form of source and/or installable code, which, upon compilation and/or installation on the computer or hardware system 500 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, field-programmable gate arrays, application-specific integrated circuits, and/or the like) might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer or hardware system (such as the computer or hardware system 500) to perform methods in accordance with various embodiments of the invention. According to a set of embodiments, some or all of the procedures of such methods are performed by the computer or hardware system 500 in response to processor 510 executing one or more sequences of one or more instructions (which might be incorporated into the operating system 540 and/or other code, such as an application program 545) contained in the working memory 535. Such instructions may be read into the working memory 535 from another computer readable medium, such as one or more of the storage device(s) 525. Merely by way of example, execution of the sequences of instructions contained in the working memory 535 might cause the processor(s) 510 to perform one or more procedures of the methods described herein.

The terms “machine readable medium” and “computer readable medium,” as used herein, refer to any medium that participates in providing data that causes a machine to operate in a specific fashion. In an embodiment implemented using the computer or hardware system 500, various computer readable media might be involved in providing instructions/code to processor(s) 510 for execution and/or might be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 525. Volatile media includes, without limitation, dynamic memory, such as the working memory 535. In some alternative embodiments, a computer readable medium may take the form of transmission media, which includes, without limitation, coaxial cables, copper wire, and fiber optics, including the wires that comprise the bus 505, as well as the various components of the communication subsystem 530 (and/or the media by which the communications subsystem 530 provides communication with other devices). In an alternative set of embodiments, transmission media can also take the form of waves (including without limitation radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer or hardware system 500. These signals, which might be in the form of electromagnetic signals, acoustic signals, optical signals, and/or the like, are all examples of carrier waves on which instructions can be encoded, in accordance with various embodiments of the invention.

The communications subsystem 530 (and/or components thereof) generally will receive the signals, and the bus 505 then might carry the signals (and/or the data, instructions, etc. carried by the signals) to the working memory 535, from which the processor(s) 505 retrieves and executes the instructions. The instructions received by the working memory 535 may optionally be stored on a storage device 525 either before or after execution by the processor(s) 510.

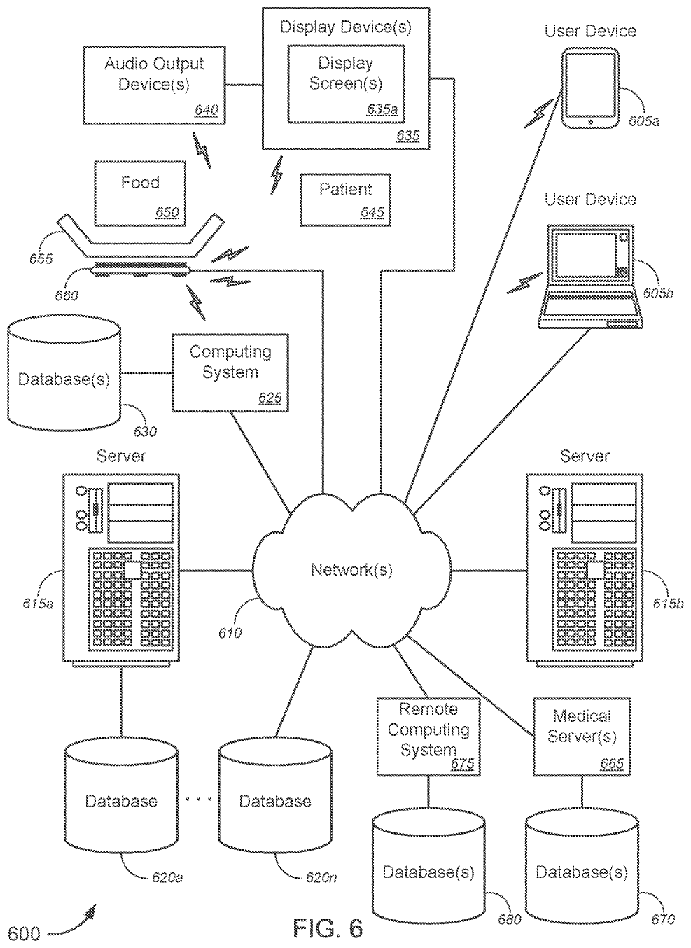

As noted above, a set of embodiments comprises methods and systems for implementing medical or medical-related diagnosis and treatment, and, more particularly, to methods, systems, and apparatuses for implementing risk assessment for suicide and treatment based on interaction with virtual clinician, food intake tracking, and/or satiety determination. FIG. 6 illustrates a schematic diagram of a system 600 that can be used in accordance with one set of embodiments. The system 600 can include one or more user computers, user devices, or customer devices 605. A user computer, user device, or customer device 605 can be a general purpose personal computer (including, merely by way of example, desktop computers, tablet computers, laptop computers, handheld computers, and the like, running any appropriate operating system, several of which are available from vendors such as Apple, Microsoft Corp., and the like), cloud computing devices, a server(s), and/or a workstation computer(s) running any of a variety of commercially-available UNIX™ or UNIX-like operating systems. A user computer, user device, or customer device 605 can also have any of a variety of applications, including one or more applications configured to perform methods provided by various embodiments (as described above, for example), as well as one or more office applications, database client and/or server applications, and/or web browser applications. Alternatively, a user computer, user device, or customer device 605 can be any other electronic device, such as a thin-client computer, Internet-enabled mobile telephone, and/or personal digital assistant, capable of communicating via a network (e.g., the network(s) 610 described below) and/or of displaying and navigating web pages or other types of electronic documents. Although the exemplary system 600 is shown with two user computers, user devices, or customer devices 605, any number of user computers, user devices, or customer devices can be supported.

Certain embodiments operate in a networked environment, which can include a network(s) 610. The network(s) 610 can be any type of network familiar to those skilled in the art that can support data communications using any of a variety of commercially-available (and/or free or proprietary) protocols, including, without limitation, TCP/IP, SNA™, IPX™, AppleTalk™, and the like. Merely by way of example, the network(s) 610 (similar to network(s) 160 FIG. 1, or the like) can each include a local area network (“LAN”), including, without limitation, a fiber network, an Ethernet network, a Token-Ring™ network, and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, the Z-Wave protocol known in the art, the ZigBee protocol or other IEEE 802.15.4 suite of protocols known in the art, and/or any other wireless protocol; and/or any combination of these and/or other networks. In a particular embodiment, the network might include an access network of the service provider (e.g., an Internet service provider (“ISP”)). In another embodiment, the network might include a core network of the service provider, and/or the Internet.

Embodiments can also include one or more server computers 615. Each of the server computers 615 may be configured with an operating system, including, without limitation, any of those discussed above, as well as any commercially (or freely) available server operating systems. Each of the servers 615 may also be running one or more applications, which can be configured to provide services to one or more clients 605 and/or other servers 615.

Merely by way of example, one of the servers 615 might be a data server, a web server, a cloud computing device(s), or the like, as described above. The data server might include (or be in communication with) a web server, which can be used, merely by way of example, to process requests for web pages or other electronic documents from user computers 605. The web server can also run a variety of server applications, including HTTP servers, FTP servers, CGI servers, database servers, Java servers, and the like. In some embodiments of the invention, the web server may be configured to serve web pages that can be operated within a web browser on one or more of the user computers 605 to perform methods of the invention.

The server computers 615, in some embodiments, might include one or more application servers, which can be configured with one or more applications accessible by a client running on one or more of the client computers 605 and/or other servers 615. Merely by way of example, the server(s) 615 can be one or more general purpose computers capable of executing programs or scripts in response to the user computers 605 and/or other servers 615, including, without limitation, web applications (which might, in some cases, be configured to perform methods provided by various embodiments). Merely by way of example, a web application can be implemented as one or more scripts or programs written in any suitable programming language, such as Java™, C, C#™ or C++, and/or any scripting language, such as Perl, Python, or TCL, as well as combinations of any programming and/or scripting languages. The application server(s) can also include database servers, including, without limitation, those commercially available from Oracle™, Microsoft™, Sybase™, IBM™, and the like, which can process requests from clients (including, depending on the configuration, dedicated database clients, API clients, web browsers, etc.) running on a user computer, user device, or customer device 605 and/or another server 615. In some embodiments, an application server can perform one or more of the processes for implementing medical or medical-related diagnosis and treatment, and, more particularly, to methods, systems, and apparatuses for implementing risk assessment for suicide and treatment based on interaction with virtual clinician, food intake tracking, and/or satiety determination, as described in detail above. Data provided by an application server may be formatted as one or more web pages (comprising HTML, JavaScript, etc., for example) and/or may be forwarded to a user computer 605 via a web server (as described above, for example). Similarly, a web server might receive web page requests and/or input data from a user computer 605 and/or forward the web page requests and/or input data to an application server. In some cases, a web server may be integrated with an application server.

In accordance with further embodiments, one or more servers 615 can function as a file server and/or can include one or more of the files (e.g., application code, data files, etc.) necessary to implement various disclosed methods, incorporated by an application running on a user computer 605 and/or another server 615. Alternatively, as those skilled in the art will appreciate, a file server can include all necessary files, allowing such an application to be invoked remotely by a user computer, user device, or customer device 605 and/or server 615.

It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 620a-620n (collectively, “databases 620”). The location of each of the databases 620 is discretionary: merely by way of example, a database 620a might reside on a storage medium local to (and/or resident in) a server 615a (and/or a user computer, user device, or customer device 605). Alternatively, a database 620n can be remote from any or all of the computers 605, 615, so long as it can be in communication (e.g., via the network 610) with one or more of these. In a particular set of embodiments, a database 620 can reside in a storage-area network (“SAN”) familiar to those skilled in the art. (Likewise, any necessary files for performing the functions attributed to the computers 605, 615 can be stored locally on the respective computer and/or remotely, as appropriate.) In one set of embodiments, the database 620 can be a relational database, such as an Oracle database, that is adapted to store, update, and retrieve data in response to SQL-formatted commands. The database might be controlled and/or maintained by a database server, as described above, for example.

According to some embodiments, system 600 might further comprise a computing system 625 and corresponding database(s) 630 (similar to computing system 105a and corresponding database(s) 110a of FIG. 1, or the like), one or more display devices 635 with corresponding display screen(s) 635a (similar to display device(s) 115 with corresponding display screen(s) 115a of FIG. 1, or the like), one or more audio output devices 640 (similar to audio output device(s) 120 of FIG. 1, or the like). System 600 might further comprise a scale 660 (similar to scale 145 of FIG. 1, or the like), one or more medical servers 665 and corresponding database(s) 670 (similar to medical server(s) 150 and corresponding database(s) 155 of FIG. 1, or the like), and remote computing system 675 and corresponding database(s) 680 (similar to computing system 105b and corresponding database(s) 110b of FIG. 1, or the like).

In operation, scale 660 might be used to measure the weight of food 650 on food container 655 during meals consumed by patient 645, with the food 650 being consumed out of the food container 655 during the meals. In this manner, the scale 660 can monitor and track the amount of food being consumed by the patient, while also tracking times of day, the number of meals per day, as well as the rate of food consumption during each meal, and any interruptions or disruptions during meals, and the like (collectively, “food intake data” or the like). The scale 660 might communicatively couple (either via wired or wireless connection), and send the food intake data, to at least one of the user device(s) 605a or 605b and/or the computing system 625, or the like. The user device(s) 605a or 605b and/or the computing system 625 might also prompt the patient 645 to enter self-reported feelings of satiety during meals, and might record such self-reported feelings of satiety. The user device(s) 605a or 605b might send the food intake and satiety data to the computing system 625, the computing system 675, and/or the medical server(s) 665 for analysis, together with data obtained during sessions in which the patient 645 interacts with a virtual clinician, as described below.

During one or more sessions, the computing system 625, the computing system 675, and/or the medical server(s) 665 (collectively, “computing system” or the like) might generate a virtual clinician capable of simulating facial expressions and body expressions (such as the virtual clinician 220, “Dr. Cecilia,” as shown in FIGS. 2B-2E and described with respect to FIGS. 3A and 3B, or the like). The generated virtual clinician would also be capable of interacting with patients to hold conversations, or the like. The computing system might cause the generated virtual clinician to interact with patient 645.

In some embodiments, causing the generated virtual clinician to interact with the patient might comprise causing, with the computing system, the generated virtual clinician to interact with patient 645, via at least one of participating in a conversation with the patient, asking the patient one or more questions, or answering one or more questions posed by the patient, where interactions between the virtual clinician and the patient 645 might be based at least in part on one or more of using, recognizing, or interpreting one or more of words, verbal expressions, statements, sentences, sentence responses, questions, or answers that are stored in a database (e.g., database(s) 630, 680, and/or 670, or the like). Alternatively, or additionally, causing the generated virtual clinician to interact with the patient might comprise one of interacting with the patient by displaying the generated virtual clinician on (display screen 635a of) display device 635 and displaying words of the virtual clinician as text on the (display screen 635a of) display device 635; interacting with the patient by displaying the generated virtual clinician on (display screen 635a of) display device 635 and presenting words of the virtual clinician via audio output device 640; or interacting with the patient by displaying the generated virtual clinician on (display screen 635a of) display device 635, presenting words of the virtual clinician via an audio output device 640, and displaying words of the virtual clinician as text on (display screen 635a of) display device 635.

Alternatively, or additionally, causing the generated virtual clinician to interact with the patient might comprise at least one of: (1) prompting the patient, with the computing system, to select a facial expression among a range of facial expressions that represents current emotions of the patient, and receiving, with the computing system, a first response from the patient, the first response comprising a selection of a facial expression that represents current emotions of the patient; (2) prompting the patient, with the computing system, to select a body posture among a range of body postures that represents current emotions of the patient, and receiving, with the computing system, a second response from the patient, the first response comprising a selection of a body posture that represents current emotions of the patient; or (3) prompting the patient, with the computing system, to select a statement regarding zest for life among a range of statements regarding zest for life that represents current thoughts of the patient regarding life and death, and receiving, with the computing system, a third response from the patient, the third response comprising a selection of a statement regarding zest for life that represents current thoughts of the patient regarding life and death; and/or the like.

Alternatively, or additionally, causing the generated virtual clinician to interact with the patient might comprise at least one of: recording video of the patient during the interaction and utilizing at least one of facial analysis, body analysis, or speech analysis to identify at least one of facial expressions of the patient, body language of the patient, or words spoken by the patient; recording audio of the patient during the interaction and utilizing speech analysis to identify words spoken by the patient; or recording words typed by the patient via a user interface device and utilizing text analysis to identify words typed by the patient; and/or the like.

During or after each session, the computing system might identify one or more flagged words or expressions spoken and/or typed by the patient during the interaction. According to some embodiments, identifying one or more flagged words or expressions spoken and/or typed by the patient during the interaction might comprise determining, with the computing system, whether words or expressions spoken or typed by the patient match predetermined flagged words and expressions that are indicative of suicide risk (such as the words and expressions depicted in FIG. 2A, or the like). The computing system might also record, to a datastore (e.g., database(s) 630, 680, and/or 670, or the like), the interactions between the virtual clinician and the patient.

While the system can track and analyze interactions and other information regarding the patient 645 during each session (i.e., performing Intra Session Data Processing), the system may also track or analyze across multiple sessions with the patient (i.e., performing Inter Session Data Processing), by compiling and analyzing historical data associated with the patient. Merely by way of example, in some cases, the historical data might include, but is not limited to, at least one of interactions between the virtual clinician and the patient during one or more prior sessions, one or more diary entries entered by the patient, one or more records containing words or expressions previous spoken or typed by the patient that match predetermined flagged words and expression that are indicative of suicide risk, one or more records containing data related to emotions of the patient during prior sessions, one or more prior suicide risk assessments for the patient, or one or more prior medical-related assessments performed on the patient, and/or the like.

In some embodiments, the computing system might analyze patient data to determine likelihood of risk of suicide by the patient. According to some embodiments, the patient data might include, without limitation, at least one of the received food intake and satiety data associated with the patient (as obtained from the scale 660 and/or the user device(s) 605a or 605b, or the like); the interactions between the virtual clinician and the patient; one or more of the received first response, the received second response, and/or the received third response; or the historical data associated with the patient; and/or the like. Based on a determination that a likelihood of risk of suicide by the patient exceeds a first predetermined threshold value, the computing system might send a message to one or more healthcare professionals (i.e., to the other of user devices 605b or 605a associated with corresponding one or more healthcare professional) regarding the likelihood of risk of suicide by the patient. Based on a determination that a likelihood of risk of suicide by the patient is below the first predetermined threshold value but exceeds a second predetermined threshold value, the computing system might send suggestions to the patient 645 (e.g., by sending the suggestions to user device(s) 605a or 605b associated with patient 645) to change eating behavior of the patient toward at least one of eating rates, food amounts, and mealtime durations that correspond to levels designed to stimulate physiological responses that evoke positive feelings for the patient.

These and other functions of the system 600 (and its components) are described in greater detail above with respect to FIGS. 1-4.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to particular structural and/or functional components for ease of description, methods provided by various embodiments are not limited to any particular structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in a particular order for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a particular structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A method, comprising:
generating, with a computing system, a virtual clinician capable of simulating facial expressions and body expressions;
causing, with the computing system, the generated virtual clinician to interact with a patient;
analyzing, with the computing system, the interactions between the virtual clinician and the patient to determine likelihood of risk of suicide by the patient; and
based on a determination that a likelihood of risk of suicide by the patient exceeds a first predetermined threshold value, sending, with the computing system, an alert message to one or more healthcare professionals regarding the likelihood of risk of suicide by the patient.

2. The method of claim 1, wherein causing the generated virtual clinician to interact with the patient comprises causing, with the computing system, the generated virtual clinician to interact with a patient, via at least one of participating in a conversation with the patient, asking the patient one or more questions, or answering one or more questions posed by the patient, wherein interactions between the virtual clinician and the patient are based at least in part on one or more of using, recognizing, or interpreting one or more of words, verbal expressions, statements, sentences, sentence responses, questions, or answers that are stored in a database.

3. The method of claim 1, further comprising:
recording, with the computing system and to a datastore, interactions between the virtual clinician and the patient.

4. The method of claim 3, wherein recording the interactions between the virtual clinician and the patient and analyzing the interactions between the virtual clinician and the patient to determine likelihood of risk of suicide by the patient are performed in real-time or near-real-time.

5. The method of claim 1, wherein causing the generated virtual clinician to interact with the patient comprises one of:
interacting with the patient by displaying the generated virtual clinician on a display device and displaying words of the virtual clinician as text on the display device;
interacting with the patient by displaying the generated virtual clinician on a display device and presenting words of the virtual clinician via an audio output device; or
interacting with the patient by displaying the generated virtual clinician on a display device, presenting words of the virtual clinician via an audio output device, and displaying words of the virtual clinician as text on the display device.

6. The method of claim 1, wherein causing the generated virtual clinician to interact with a patient comprises at least one of:
prompting the patient, with the computing system, to select a facial expression among a range of facial expressions that represents current emotions of the patient, and receiving, with the computing system, a first response from the patient, the first response comprising a selection of a facial expression that represents current emotions of the patient;
prompting the patient, with the computing system, to select a body posture among a range of body postures that represents current emotions of the patient, and receiving, with the computing system, a second response from the patient, the first response comprising a selection of a body posture that represents current emotions of the patient; or
prompting the patient, with the computing system, to select a statement regarding zest for life among a range of statements regarding zest for life that represents current thoughts of the patient regarding life and death, and receiving, with the computing system, a third response from the patient, the third response comprising a selection of a statement regarding zest for life that represents current thoughts of the patient regarding life and death;
wherein analyzing the interactions between the virtual clinician and the patient to determine likelihood of risk of suicide by the patient comprises analyzing, with the computing system, the interactions between the virtual clinician and the patient and at least one of the received first response, the received second response, or the received third response, to determine likelihood of risk of suicide by the patient.

7. The method of claim 1, wherein causing the generated virtual clinician to interact with the patient comprises at least one of:
recording video of the patient during the interaction and utilizing at least one of facial analysis, body analysis, or speech analysis to identify at least one of facial expressions of the patient, body language of the patient, or words spoken by the patient;
recording audio of the patient during the interaction and utilizing speech analysis to identify words spoken by the patient; or
recording words typed by the patient via a user interface device and utilizing text analysis to identify words typed by the patient.

8. The method of claim 1, wherein analyzing the interactions between the virtual clinician and the patient to determine likelihood of risk of suicide by the patient comprises:
determining, with the computing system, whether words or expressions spoken or typed by the patient match predetermined flagged words and expressions that are indicative of suicide risk; and
determining, with the computing system, likelihood of risk of suicide by the patient, based at least in part on a determination that words or expressions spoken or typed by the patient match predetermined flagged words and expressions that are indicative of suicide risk.

9. The method of claim 1, wherein analyzing the interactions between the virtual clinician and the patient to determine likelihood of risk of suicide by the patient comprises analyzing, with the computing system, the interactions between the virtual clinician and the patient and historical data associated with the patient to determine likelihood of risk of suicide by the patient, wherein the historical data comprises at least one of interactions between the virtual clinician and the patient during one or more prior sessions, one or more diary entries entered by the patient, one or more records containing words or expressions previous spoken or typed by the patient that match predetermined flagged words and expression that are indicative of suicide risk, one or more records containing data related to emotions of the patient during prior sessions, one or more prior suicide risk assessments for the patient, or one or more prior medical-related assessments performed on the patient.

10. The method of claim 1, wherein analyzing the interactions between the virtual clinician and the patient to determine likelihood of risk of suicide by the patient comprises utilizing at least one of artificial intelligence functionality or machine learning functionality to determine likelihood of risk of suicide by the patient.

11. The method of claim 1, further comprising:
receiving, with the computing system, food intake and satiety data associated with the patient, the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals;
wherein analyzing the interactions between the virtual clinician and the patient to determine likelihood of risk of suicide by the patient comprises analyzing, with the computing system, at least one of the interactions between the virtual clinician and the patient or the received food intake and satiety data associated with the patient, to determine likelihood of risk of suicide by the patient.

12. The method of claim 11, wherein the food intake and satiety data associated with the patient are received from at least one of a communications-enabled scale that is used to measure weight of food on a food container during meals consumed by the patient where the food is consumed out of the food container during the meals, a user device that is communicatively coupled to the communications-enabled scale, or the user device that records self-reported feelings of satiety from the patient during meals.

13. The method of claim 11, further comprising:
based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.

14. An apparatus, comprising:
at least one processor; and
a non-transitory computer readable medium communicatively coupled to the at least one processor, the non-transitory computer readable medium having stored thereon computer software comprising a set of instructions that, when executed by the at least one processor, causes the apparatus to:
generate a virtual clinician capable of simulating facial expressions and body expressions;
cause the generated virtual clinician to interact with a patient;
analyze the interactions between the virtual clinician and the patient to determine likelihood of risk of suicide by the patient; and
based on a determination that a likelihood of risk of suicide by the patient exceeds a first predetermined threshold value, send an alert message to one or more healthcare professionals regarding the likelihood of risk of suicide by the patient.

15. The apparatus of claim 14, wherein causing the generated virtual clinician to interact with the patient comprises causing the generated virtual clinician to interact with a patient, via at least one of participating in a conversation with the patient, asking the patient one or more questions, or answering one or more questions posed by the patient, wherein interactions between the virtual clinician and the patient are based at least in part on one or more of words, verbal expressions, statements, sentences, sentence responses, questions, or answers that are stored in a database.

16. The apparatus of claim 14, wherein analyzing the interactions between the virtual clinician and the patient to determine likelihood of risk of suicide by the patient comprises:
determining whether words or expressions spoken or typed by the patient match predetermined flagged words and expressions that are indicative of suicide risk; and
determining likelihood of risk of suicide by the patient, based at least in part on a determination that words or expressions spoken or typed by the patient match predetermined flagged words and expressions that are indicative of suicide risk.

17. The apparatus of claim 14, wherein the set of instructions, when executed by the at least one processor, further causes the apparatus to:
receive food intake and satiety data associated with the patient, the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals;
wherein analyzing the interactions between the virtual clinician and the patient to determine likelihood of risk of suicide by the patient comprises analyzing at least one of the interactions between the virtual clinician and the patient or the received food intake and satiety data associated with the patient, to determine likelihood of risk of suicide by the patient.

18. A system, comprising:
a computing system, comprising:
at least one first processor; and
a first non-transitory computer readable medium communicatively coupled to the at least one first processor, the first non-transitory computer readable medium having stored thereon computer software comprising a first set of instructions that, when executed by the at least one first processor, causes the computing system to:
generate a virtual clinician capable of simulating facial expressions and body expressions;
cause the generated virtual clinician to interact with a patient;
analyze the interactions between the virtual clinician and the patient to determine likelihood of risk of suicide by the patient; and
based on a determination that a likelihood of risk of suicide by the patient exceeds a first predetermined threshold value, send an alert message to one or more healthcare professionals regarding the likelihood of risk of suicide by the patient.

19. The system of claim 18, further comprising:
a scale that is used to measure weight of food on a food container during meals consumed by the patient, where the food is consumed out of the food container during the meals; and
a user device associated with the user, and communicatively coupled to the scale, the user device comprising:
at least one second processor; and
a second non-transitory computer readable medium communicatively coupled to the at least one second processor, the second non-transitory computer readable medium having stored thereon computer software comprising a second set of instructions that, when executed by the at least one second processor, causes the user device to:
receive food intake data associated with the patient from the scale, wherein the food intake and satiety data comprises at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, or information regarding occurrence of any displaced behaviors during a meal;
prompt the patient to enter self-reported feelings of satiety from the patient during meals and receive satiety data from the patient; and
send food intake and satiety data associated with the patient to the computing system;
wherein analyzing the interactions between the virtual clinician and the patient to determine likelihood of risk of suicide by the patient comprises analyzing at least one of the interactions between the virtual clinician and the patient or the received food intake and satiety data associated with the patient, to determine likelihood of risk of suicide by the patient.

20. The system of claim 19, wherein the user device comprises one of a tablet computer, a smart phone, a mobile phone, a laptop computer, a desktop computer, or a dedicated food intake tracking device.

21. A method, comprising:
receiving, with a computing system, food intake and satiety data associated with the patient;
analyzing, with the computing system, the food intake and satiety data associated with the patient to determine likelihood of risk of suicide by the patient; and
based on a determination that a likelihood of risk of suicide by the patient exceeds a first predetermined threshold value, sending, with the computing system, an alert message to one or more healthcare professionals regarding the likelihood of risk of suicide by the patient.

22. The method of claim 21, wherein the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals.

23. The method of claim 21, wherein the food intake and satiety data associated with the patient are received from at least one of a communications-enabled scale that is used to measure weight of food on a food container during meals consumed by the patient where the food is consumed out of the food container during the meals, a user device that is communicatively coupled to the communications-enabled scale, or the user device that records self-reported feelings of satiety from the patient during meals.

24. The method of claim 21, further comprising:
based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.

25. A system, comprising:
a scale that is used to measure weight of food on a food container during meals consumed by the patient, where the food is consumed out of the food container during the meals; and
a user device associated with the user, and communicatively coupled to the scale, the user device comprising:
at least one first processor; and
a first non-transitory computer readable medium communicatively coupled to the at least one first processor, the first non-transitory computer readable medium having stored thereon computer software comprising a first set of instructions that, when executed by the at least one first processor, causes the user device to:
receive food intake data associated with the patient from the scale, wherein the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, or information regarding occurrence of any displaced behaviors during a meal;
prompt the patient to enter self-reported feelings of satiety from the patient during meals and receive satiety data from the patient; and
send food intake and satiety data associated with the patient to a computing system;
the computing system, comprising:
at least one second processor; and
a second non-transitory computer readable medium communicatively coupled to the at least one second processor, the second non-transitory computer readable medium having stored thereon computer software comprising a second set of instructions that, when executed by the at least one second processor, causes the computing system to:
receive the food intake and satiety data associated with the patient;
analyze the food intake and satiety data associated with the patient to determine likelihood of risk of suicide by the patient; and
based on a determination that a likelihood of risk of suicide by the patient exceeds a first predetermined threshold value, send an alert message to one or more healthcare professionals regarding the likelihood of risk of suicide by the patient.

26. A method, comprising:
generating, with a computing system, a virtual clinician capable of simulating facial expressions and body expressions;
causing, with the computing system and using a display device and an audio output device, the generated virtual clinician to interact with a patient, via at least one of participating in a conversation with the patient, asking the patient one or more questions, or answering one or more questions posed by the patient, wherein interactions between the virtual clinician and the patient are based at least in part on one or more of words, verbal expressions, statements, sentences, sentence responses, questions, or answers that are stored in a database;
recording, with the computing system and to a datastore, interactions between the virtual clinician and the patient;
prompting the patient, with the computing system and using the display device viewable by the patient and the audio output device, to select a facial expression among a range of facial expressions that represents current emotions of the patient;
receiving, with the computing system, a first response from the patient, the first response comprising a selection of a facial expression that represents current emotions of the patient;
prompting the patient, with the computing system and using the display device viewable by the patient and the audio output device, to select a body posture among a range of body postures that represents current emotions of the patient;
receiving, with the computing system, a second response from the patient, the second response comprising a selection of a body posture that represents current emotions of the patient;
prompting the patient, with the computing system and using the display device viewable by the patient and the audio output device, to select a statement regarding zest for life among a range of statements regarding zest for life that represents current thoughts of the patient regarding life and death;
receiving, with the computing system, a third response from the patient, the third response comprising a selection of a statement regarding zest for life that represents current thoughts of the patient regarding life and death;
receiving, with the computing system, food intake and satiety data associated with the patient, the food intake and satiety data comprising at least one of information regarding amount of food consumed per meal, information regarding changes in amount of food consumed per meal, information regarding rate of food consumption, information regarding changes in rate of food consumption, information regarding eating patterns related to rate of food consumption, information regarding normal meal consumption characteristics for the patient, information regarding amount of deviation from normal meal consumption characteristics for the patient, information regarding occurrence of any displaced behaviors during a meal, or information regarding self-reported feelings of satiety from the patient corresponding to individual meals;
analyzing, with the computing system, at least one of the recorded interactions between the virtual clinician and the patient, the received first response, the received second response, the received third response, or the received food intake and satiety data associated with the patient, to determine likelihood of risk of suicide by the patient;
based on a determination that a likelihood of risk of suicide by the patient exceeds a first predetermined threshold value, sending, with the computing system, a message to one or more healthcare professionals regarding the likelihood of risk of suicide by the patient; and
based on a determination that a likelihood of risk of suicide by the patient is below the first predetermined threshold value but exceeds a second predetermined threshold value, sending, with the computing system, suggestions to the patient to change eating behavior of the patient toward at least one of eating rates, food amounts, and mealtime durations that correspond to levels designed to stimulate physiological responses that evoke positive feelings for the patient.
